SEQUENCE LISTING (15 sequences)

SEQ ID NO: 1 - protein, 154 aa, Unknown
  Aquifex aeolicus
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 2 - DNA, 465 bp, Unknown
  Aquifex aeolicus
atgcaaatttacgaagggaaactaaccgctgaagggctgaggttcggtatagtggcttccaggttcaaccacgcactcgtggatagactagttgagggagctatagactgcatagtaagacacgggggaagggaagaagacataacgctcgttagagtgccgggctcctgggaaattcccgtggctgcgggagagcttgcgagaaaagaggacatagacgctgtgatagcgataggagttctaataaggggggctactccccactttgattacatagcctctgaagtgtcaaaagggcttgcgaacctttccttagaactgagaaaacccataaccttcggtgttataactgcggacaccttggagcaggcgatagaaagggcgggaacaaagcacgggaataagggctgggaagctgcactttccgcaatagaaatggcaaacttatttaagagtctgagatga

SEQ ID NO: 3 - protein, 139 aa, Artificial Sequence
  Synthetic
MSYYHHHHHHDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 4 - protein, 13 aa, Artificial Sequence
  Synthetic
AHIVMVDAYKPTK

SEQ ID NO: 5 - protein, 1255 aa, Unknown
  SARS-CoV-1
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 6 - protein, 222 aa, Unknown
  SARS-CoV-1
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 7 - protein, 1273 aa, Unknown
  SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 223 aa, Unknown
  SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 9 - protein, 1353 aa, Unknown
  MERS-CoV
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSTVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 10 - protein, 122 aa, Unknown
  MERS-CoV
ECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKC

SEQ ID NO: 11 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 107 aa, Artificial Sequence [sequence omitted]

SEQ ID NO: 15 - protein, 673 aa, Artificial Sequence
  Synthetic
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

(19) United States
(12) Patent Application Publication
(10) Pub. No.: US 2024/0115693 A1
(43) Pub. Date: Apr. 11, 2024

(54) SARS-COV-2 ANTIGEN NANOPARTICLES AND USES THERE OF

(71) Applicants: Children's Medical Center Corporation, Boston, MA (US); Dana-Farber Cancer Institute, Inc., Boston, MA (US)

(72) Inventors: Francesco Borriello, Arlington, MA (US); Ofer Levy, Cambtidge, MA (US); David J. Dowling, Milton, MA (US); Sirano Dhe-Paganon, Holliston, MA (US); Hyuk-Soo Seo, Boston, MA (US)

(73) Assignees: Children's Medical Center Corporation, Boston, MA (US); Dana-Farber Cancer Institute, Inc., Boston, MA (US)

(21) Appl. No.: 18/572,163

(22) PCT Filed: Jun. 23, 2022
(86) PCT No.: PCT/US2022/034673

§ 371 Date: Dec. 19, 2023

Related U.S. Application Data
(60) Provisional application No. 63/215,312, filed on Jun. 25, 2021.

(51) Int. Cl. A61K 39/215; A61K 9/51; A61P 31/14

(52) U.S. Cl. CPC A61K 39/215; A61K 9/5169; A61P 31/14; A61K 2039/64

(57) ABSTRACT

Provided herein are nanoparticles which enhance the immunogenicity of antigens for use in Beta coronavirus (e.g., MERS-CoV, SARS-CoV-1, or SARS-CoV-2) vaccines, as well as immunogenic compositions comprising the antigen nanoparticles and additional adjuvants for further enhancing immunogenicity.

### RELATED APPLICATIONS

This application claims priority under 35 U.S.C. § 119(e) to U.S. Provisional Application No. 63/215,312, entitled “SARS-COV-2 ANTIGEN NANOPARTICLES AND USES THERE OF”, filed on Jun. 25, 2021, the contents of which are incorporated herein by reference in their entirety.

### GOVERNMENT SUPPORT

This disclosure was made with government support under Adjuvant Discovery Program contract number HHSN272201400052C, grant number 75N93019C00044, awarded by the National Institutes of Health (NIH). The government has certain rights in this invention.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Jun. 23, 2022, is named C123370217WO00-SEQ-ZJG and is 58,236 bytes in size.

### BACKGROUND

Severe acute respiratory syndrome-related coronavirus (SARS-CoV) is a member of the genus Betacoronavirus and subgenus Sarbecoronavirus, and is a species of coronavirus that infects humans, bats and certain other mammals. It is an enveloped positive-sense single-stranded RNA virus that enters its host cell by binding to the angiotensin-converting enzyme 2 (ACE2) receptor.

Two strains of the virus have caused outbreaks of severe respiratory diseases in humans: severe acute respiratory syndrome coronavirus (SARS-CoV or SARS-CoV-1), which caused the 2002-2004 outbreak of severe acute respiratory syndrome (SARS), and severe acute respiratory syndrome coronavirus 2 (SARS-CoV-2), which is causing the 2019-2020 pandemic of coronavirus disease 2019 (COVID-19). There are hundreds of other strains of SARS-CoV.

### SUMMARY

Discovery, development and implementation of safe and effective vaccines will be key to addressing the SARS-CoV-2 pandemic. Immunization of distinct vulnerable populations such as the elderly may result in sub-optimal responses, often requiring multiple booster doses and can be limited by waning immunity. The efficiency and efficacy of vaccination can frequently be improved by optimizing the immunogenicity of vaccine antigens and may be achieved, for instance, by multimerizing poorly immunogenic antigens. Adjuvantation is also key to enhancing vaccine-induced immunity, as adjuvants can enhance, prolong, and modulate immune responses to vaccinal antigens to maximize protective immunity. Optimization of antigen immunogenicity and adjuvantation may potentially enable effective immunization in otherwise vulnerable populations (e.g., in the very young and the elderly or for diseases lacking effective vaccines).

Some aspects of the present disclosure provide a nanoparticle comprising a multimeric protein scaffold comprising lumazine synthase and a protein antigen from a Beta coronavirus. In some embodiments, the lumazine synthase is that from Aquifex aeolicus. In some embodiments, the multimeric protein scaffold comprises at least 60 subunits of lumazine synthase.

In some embodiments, the Beta coronavirus is MERS-CoV, SARS-CoV-1, or SARS-CoV-2. In some embodiments, the Beta coronavirus protein antigen is a MERS-CoV spike protein, a SARS-CoV-1 spike protein, or a SARS-CoV-2 spike protein. In some embodiments, the Beta coronavirus protein antigen is a protein domain from a MERS-CoV, SARS-CoV-1, or SARS-CoV-2 protein. In some embodiments, the Beta coronavirus protein antigen is a MERS-CoV spike protein receptor binding domain (RBD), a SARS-CoV-1 spike protein RBD, or a SARS-CoV-2 spike protein RBD.

In some embodiments, the lumazine synthase and the Beta coronavirus protein antigen are covalently linked. In some embodiments, the lumazine synthase and the Beta coronavirus protein antigen are covalently linked through a covalent bond formed between SEQ ID NO: 3 (SpyCatcher) and SEQ ID NO: 4 (SpyTag). In some embodiments, The Beta coronavirus protein antigen is displayed on the surface of the nanoparticle.

In some embodiments, the nanoparticle enhances an immune response against the Beta coronavirus protein antigen when administered to a subject, compared to when the protein antigen is administered alone (i.e., in the absence of the nanoparticle). In some embodiments, the nanoparticle enhances the production of antigen-specific antibodies when administered to a subject, compared to when the protein antigen is administered alone (i.e., in the absence of the nanoparticle). In some embodiments, the antigen-specific antibodies comprise immunoglobulin G (IgG). In some embodiments, the IgG is a subclass 1 IgG (IgG1) or a subclass 2 IgG (IgG2). In some embodiments, the antigen-specific antibodies are neutralizing antibodies against a variant of MERS-CoV, SARS-CoV-1, or SARS-CoV-2. In some embodiments, the antigen specific antibodies are neutralizing antibodies against wild-type SARS-CoV-2, B.1.1.7 SARS-CoV-2, or B.1.351 SARS-CoV-2. In some embodiments, the nanoparticle prolongs a protective effect against the Beta coronavirus protein antigen in a subject, compared to when the Beta coronavirus protein antigen is administered alone (i.e., in the absence of the nanoparticle). In some embodiments, the subject is human.

Some aspects of the present disclosure provide a composition comprising the nanoparticle. In some embodiments, the composition further comprises a pharmaceutically acceptable carrier.

Some aspects of the present disclosure provide a composition comprising the nanoparticle and a squalene-based oil in water emulsion (OIW) or a liposomal adjuvant. In some embodiments, the squalene-based OIW comprises an emulsion of sorbitan trioleate, squalene, and polysorbate 80 (AddaVax) or an emulsion of DL-α-tocopherol, squalene, and polysorbate 80 (AddaS03). In some embodiments, the liposomal adjuvant comprises 3-O-desacyl-4′-monophosphoryl lipid A, saponin QS-21, dioleoyl phosphatidylcholine (DOPC), and cholesterol (AS01B). In some embodiments, the composition comprises a second adjuvant. In some embodiments, the composition comprises a second adjuvant that is a Toll-like receptor (TLR) agonist. In some embodiments, the composition is a vaccine composition.

Some aspects of the present disclosure provide a method for enhancing an immune response in a subject in need thereof, comprising administering to the subject an effective amount of the nanoparticle, a composition comprising the nanoparticle, or a composition comprising the nanoparticle and a squalene-based OIW or liposomal adjuvant. Further aspects of the present disclosure provide a method for treating a disease or reducing the risk of a disease in a subject, comprising administering to the subject an effective amount of the nanoparticle, a composition comprising the nanoparticle, or a composition comprising the nanoparticle and a squalene-based OIW or liposomal adjuvant.

In some embodiments, the disease is a disease caused by a Beta coronavirus (e.g., MERS-CoV, SARS-CoV-1, or SARS-CoV-2). In some embodiments, the disease is Middle East Respiratory Syndrome (MERS), Sudden Acute Respiratory Syndrome (SARS), or Coronavirus Disease 2019 (COVID-19).

In some embodiments, the subject is a human neonate, a human infant, a human adult, or an elderly human. In some embodiments, the subject is a human adult or elder. In some embodiments, the administration occurs when the subject is more than 65 years of age. In some embodiments, the subject is immunocompromised, immunosenescent, has a chronic illness, is malnourished, or is frail

In some embodiments, the administration is intravenous, intramuscular, intradermal, oral, topical, intranasal, or sublingual. In some embodiments, the administration occurs more than once. In some embodiments, the administration elicits an immune response to a Beta coronavirus protein antigen in the subject. In some embodiments, the immune response comprises an innate immune response or an adaptive immune response. In some embodiments, the administration elicits the production of one or more pro-inflammatory cytokines in the subject. In some embodiments, the administration elicits the production of one or more of CSF-2, IL-6, and CXCL1. In some embodiments, the administration elicits the expression of one or more interferon (IFN)-sensing genes in the subject. In some embodiments, the administration elicits the expression of type I IFN-stimulated genes. In some embodiments, the IFN-stimulated genes are one or more of CXCL19, IFIT2, and RSAD2. In some embodiments, the administration enhances antigen retention in draining lymph nodes of the subject. In some embodiments, the administration elicits the production of antigen-specific antibodies in the subject. In some embodiments, the antigen-specific antibodies comprise immunoglobulin G (IgG). In some embodiments, the IgG is a subclass 1 IgG (IgG1) or a subclass 2 IgG (IgG2). In some embodiments, the antigen-specific antibodies are neutralizing antibodies against a variant of MERS-CoV, SARS-CoV-1, or SARS-CoV-2. In some embodiments, the antigen specific antibodies are neutralizing antibodies against wild-type SARS-CoV-2, B.1.1.7 SARS-CoV-2, or B.1.351 SARS-CoV-2.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1F: A lumazine synthase nanoparticle scaffold enables efficient RBD display. FIG. 1A: SDS-PAGE analysis under reducing conditions of RBD expressing SpyCatcher (RBD-Catch), lumazine synthase expressing SpyTag (LuS-Tag), RBD nanoparticle (RBD-NP) as well as native RBD and Spike proteins. FIGS. 1B-1C: Transmission electron microscopy (FIG. 1B) and dynamic light scattering (FIG. 1C) analyses of RBD-NP. FIGS. 1D-1F: ELISA plates were coated with RBD, Spike, RBD-NP, and LuS-Tag at 1 mg/ml (FIG. 1D), 5 mg/ml (FIG. 1E) or 0.5 mg/ml (FIG. 1F). Binding of recombinant human ACE2 (hACE2) or anti-RBD H4 and CR3022 antibody clones tested at multiple concentrations was expressed as optical density (OD) at 450 nm or area under the curve (AUC). N=3-6 experiments. * and ** respectively indicate p≤0.05 and 0.01. Statistical significance was determined by one-way ANOVA corrected for multiple comparisons. Comparisons among experimental groups are indicated as shaded.

FIGS. 2A-2C: RBD nanoparticle demonstrates superior immunogenicity to Spike or monomeric RBD in mice. 3-month-old BALB/c mice were injected with PBS or immunized with the indicated doses of RBD, Spike or RBD nanoparticle (RBD-NP), alone or formulated with AddaVax on day 0 (prime) and 14 (boost). Anti-RBD IgG (FIG. 2A), IgG1 (FIG. 2B) and IgG2a (FIG. 2C) antibody titers were assessed in serum samples collected on days 14 (pre-boost) and 28. Dotted lines indicate lower limit of detection. N=7-10 mice per group. * and ** respectively indicate p≤0.05 and 0.01 for comparisons among RBD, Spike and RBD-NP in the same adjuvant formulation group (−AddaVax or +AddaVax). #and ##respectively indicate p≤0.05 and 0.01 for comparisons of same antigen groups between the two adjuvant formulation groups (−AddaVax vs +AddaVax). Statistical significance was determined by two-way ANOVA corrected for multiple comparisons after Log-transformation of the raw data. Comparisons among experimental groups are indicated as shaded.

FIGS. 3A-3B: Immunization with RBD nanoparticle induces robust SARS-CoV-2 neutralizing titers at all doses tested. 3-month-old BALB/c mice were immunized as in FIGS. 2A-2C. Serum levels of anti-RBD neutralizing antibodies were assessed on day 28 by SARS-CoV-2 surrogate (FIG. 3A) and conventional (FIG. 3B) virus neutralization tests. The dotted line indicates lower limit of detection. N=7-10 mice per group. * and ** respectively indicate p≤0.05 and 0.01 for comparisons among RBD, Spike and RBD-NP in the same adjuvant formulation group (−AddaVax or +AddaVax). #and ##respectively indicate p≤0.05 and 0.01 for comparisons of same antigen groups between the two adjuvant formulation groups (−AddaVax vs +AddaVax). Statistical significance was determined by two-way ANOVA corrected for multiple comparisons. Data shown in (FIG. 3B) were Log-transformed before the analysis. Comparisons among experimental groups are indicated as shaded.

FIGS. 4A-4J: Immunogenicity of adjuvanted RBD nanoparticle in young and aged mice. Young (3-month-old, FIGS. 4A-4E) and aged (14-month-old, FIGS. 4F-4J) BALB/c mice were immunized as in FIGS. 2A-2C with PBS, RBD nanoparticle (RBD-NP) alone or formulated with AddaVax, AddaS03 or AS01B. Serum samples were collected on Day 28 to assess anti-RBD IgG (FIGS. 4A and 4F), IgG1 (FIGS. 4B and 4G), IgG2a (FIGS. 4C and 4H) antibody titers as well as anti-RBD neutralizing activity by surrogate (FIGS. 4D and 4I) and conventional (FIGS. 4E and 4J) virus neutralization tests. Dotted lines indicate lower limit of detection. N=10 (FIGS. 4A-4I) or 5 (FIG. 4J) mice per group. * and ** respectively indicate p≤0.05 and 0.01. Statistical significance was determined by one-way ANOVA corrected for multiple comparisons. Data shown in (FIGS. 4A-4C, 4E-4H, and 4J) were Log-transformed before the analysis. Comparisons among experimental groups are indicated as shaded.

FIGS. 5A-5E: Immunization with adjuvanted RBD nanoparticle completely protects aged mice from SARS-CoV-2 challenge. Aged (14-month old) BALB/c mice were immunized as in FIGS. 2A-2C with PBS, RBD nanoparticle (RBD-NP) alone or formulated with AddaVax, AddaS03 or AS01B. Seven weeks after the final immunization, mice were infected with 10³ plaque-forming units (PFU) of mouse-adapted SARS-CoV-2 and monitored for up to 4 days for weight loss. Daily weights (FIG. 5A) and survival rates (FIG. 5B) of infected mice are shown. FIGS. 5C-5E: On Day 4, mice were sacrificed and lungs were collected to assess viral titers (FIG. 5C), hematoxylin and eosin-stained lung images (FIG. 5D), and gene expression profiles shown as relative expression compared to Rlp13a (FIG. 5E). N=8-10 per group except for the AS01B group (N=4). Results in FIG. 5A represent mean±SEM. Data were compared to the PBS group by Kruskal-Wallis test corrected for multiple comparisons. Data shown in FIGS. 5C and 5E were log-transformed and analyzed by one-way ANOVAs corrected for multiple comparisons, in comparison to PBS and RBD-NP groups indicated as shaded. FIG. 5D shows representative lung images. Each symbol represents an individual sample. *, **, ***, and **** respectively indicate p<0.05, 0.01, 0.001, and 0.0001.

FIGS. 6A-6B: Adjuvanted RBD nanoparticle formulations induce cross-neutralizing antibodies. Young (3-month-old; FIG. 6A) and aged (14-month-old; FIG. 6B) BALB/c mice were immunized as in FIGS. 2A-2C with RBD nanoparticle (RBD-NP) formulated with AddaVax, AddaS03 or AS01B. Serum samples were collected on Day 28 to assess neutralizing titers (NT50) against SARS-CoV-2 wild type (WT), B.1.17 or B.1.351 pseudoviruses. Dotted lines indicate lower limit of detection. N=5 mice per group, with each dot representing an individual sample. Numbers indicate geometric mean titers for each experimental group.

FIGS. 7A-7D: Adjuvanted RBD-NP enhances CD4+ T cell responses in young and aged mice. Young adult (FIGS. 7A and 7B) and aged (FIGS. 7C and 7D) BALB/c mice (3- and 11-month-old, respectively) were immunized as in FIGS. 2A-2C with PBS, RBD nanoparticle (RBD-NP) alone or RBD-NP formulated with AddaVax, AddaS03, or AS01B. Splenocytes were collected 4 weeks after the final immunization and stimulated with a SARS-CoV-2 spike RBD peptide pool and expression of intracellular interferon-γ (IFNγ), TNF, IL-2, and IL-4/5 among CD4+ (FIGS. 7A and 7C) and CD8+ (FIGS. 8B and 7D) T cells were determined by flow cytometry. N=8-10 per group. Data were log-transformed and analyzed by one-way ANOVAs corrected for multiple comparisons. *, **, *** and **** respectively indicate p<0.05, 0.01, 0.001, and 0.0001. Each symbol represents an individual sample.

FIGS. 8A-8B: Gene signatures induced by AddaVax at the injection site (muscle) and the draining lymph node. Young (3-month-old) BALB/c mice were injected with PBS or AddaVax. 24 hours later muscle tissue at the injection sites (FIG. 8A) and dLNs (FIG. 8B) were collected to assess gene expression profiles by qPCR. Results are reported as relative expression compared to Rlp13a. N=4 mice per group. * and ** respectively indicate p≤0.05 and 0.01. Statistical significance was determined by Student's t-test after Log-transformation of the raw data. Comparisons between experimental groups are indicated as shaded.

FIG. 9: RBD nanoparticle (RBD-NP) harbors multiple glycosylation patterns. SDS-PAGE analysis of RBD expressing SpyCatcher (RBD-Catch) and RBD nanoparticle (RBD-NP) with or without Peptide-N-Glycosidase F (PNGase) treatment. Representative result of n=2 experiments is shown.

FIG. 10: RBD nanoparticle is stable under multiple storage conditions. ELISA plates were coated with RBD nanoparticles that underwent 1 (F/T×1) or 5 (F/T×5) freeze/thaw cycles, or stored for 1 week at 4° C. (4° C.—1 wk) or room temperature (RT—1 wk). Binding of recombinant human ACE2 (hACE2) or anti-RBD H4 and CR3022 antibody clones was expressed as optical density (OD) at 450 nm or area under the curve (AUC). N=4 experiments. Statistical significance was determined by one-way ANOVA corrected for multiple comparisons.

FIGS. 11A-11B: RBD nanoparticle competes with SARS-CoV-2 in vitro. Vero cells were infected with SARS-CoV-2 in the presence or absence of RBD, Spike and RBD nanoparticle (RBD-NP) tested at multiple concentrations. Results are expressed as percentage of non-infected cells. For each protein a non-linear curve was fitted and used to calculate IC₅₀ (FIG. 11A) and area under the curve (FIG. 11B). N=2 experiments.

FIG. 12: Anti-RBD antibodies elicited by RBD nanoparticle immunization recognize native RBD on Spike. Anti-Spike IgG, IgG1 and IgG2a antibody titers were assessed in serum samples collected on Day 28 as indicated in FIG. 2. Dotted lines indicate lower limit of detection. N=10 mice per group. ** indicates p≤0.01. Statistical significance was determined by one-way ANOVA corrected for multiple comparisons after Log-transformation of the raw data. Comparisons among experimental groups are indicated as shaded.

FIGS. 13A-13B: RBD nanoparticle is immunogenic in multiple mouse strains. 3-month-old C57BL/6 (FIG. 13A) and CD-1 (FIG. 13B) mice were injected with PBS or immunized with 0.3 mg RBD nanoparticle (RBD-NP), alone or formulated with AddaVax on Day 0 (prime) and Day 14 (boost). Anti-RBD IgG, IgG1 and IgG2a antibody titers were assessed in serum samples collected on Day 28. Dotted lines indicate lower limit of detection. N=5 mice per group. * and ** respectively indicate p≤0.05 and 0.01. Statistical significance was determined by one-way ANOVA corrected for multiple comparisons after Log-transformation of the raw data. Comparisons among experimental groups are indicated as shaded.

FIG. 14: Aged mice demonstrate reduced anti-RBD antibody response upon immunization. Comparisons of anti-RBD IgG, IgG1 and IgG2a antibody titers between immunized young and aged mice as reported in FIGS. 4A-4J. N=10 mice per group. * and ** respectively indicate p≤0.05 and 0.01. Statistical significance was determined by two-way ANOVA corrected for multiple comparisons after Log-transformation of the raw data. Comparisons among experimental groups are indicated as shaded.

FIG. 15: Flow cytometry gating strategy. Flow cytometry plots showing the gating strategy applied to identify RBD-specific CD4+ and CD8+ T cell responses after stimulating splenocytes with phorbol myristate acetate (PMA) and ionomycin.

FIG. 16: AddaVax promotes antigen retention in the draining lymph node. 3-month-old BALB/c mice were injected intramuscularly with PBS, R-PE, R-PE formulated with AddaVax. 24 hours later draining lymph node were collected, homogenized in water and fluorescence was measured in cleared supernatants. Results are expressed as arbitrary units (A.U.) of fluorescence. N=12 mice per group. ** indicates p≤0.01. Statistical significance was determined by one-way ANOVA corrected for multiple comparisons after Log-transformation of the raw data. Comparisons among experimental groups are indicated as shaded.

FIG. 17: Adjuvantation demonstrates a favorable reactogenicity profile. Young (3-month-old) BALB/c mice were injected with PBS, AS01B or AddaVax. Serum samples were collected 24 hours later to assess cytokine and chemokine production by multiplexing bead array. Results are reported as mean±SEM. Statistical significance was determined by Kruskal-Wallis test corrected for multiple comparisons. N=4-5 mice per group. *, ** and *** respectively indicate p<0.05, 0.01 and 0.001.

### DETAILED DESCRIPTION OF CERTAIN EMBODIMENTS

Human immunity is crucial to both health and illness, playing key roles in multiple major diseases including infectious diseases, allergy and cancer. Infectious diseases are a leading cause of morbidity and mortality among both society's youngest and oldest members. SARS-coronavirus-2 (SARS-CoV-2), the causal agent of COVID-19, first emerged in late 2019 in China. It has infected almost 175 million individuals and caused >3,700,000 deaths globally, especially in the elderly population. Discovery, development and implementation of safe and effective vaccines will be key to addressing the SARS-CoV-2 pandemic.

Immunization of distinct vulnerable populations such as the elderly may result in sub-optimal responses, often requiring multiple booster doses and can be limited by waning immunity over time. A key approach to enhancing the efficacy of vaccinations is the development of antigens that offer enhanced immunogenicity, especially in vulnerable populations, thereby reducing the need for administration of multiple vaccines to achieve immunity and/or improving the duration of effective immunity in the recipient. Such development may improve upon the immunogenicity of vaccinal antigens which are only weakly immunogenic or are typically unreliable for eliciting an immune response when administered to subjects. Additionally, adjuvantation is another major approach for enhancing vaccine-induced immunity. Adjuvants can enhance, prolong, and modulate immune responses to vaccinal antigens to maximize protective immunity, and may potentially enable effective immunization in vulnerable populations (e.g., in the very young and the elderly or for diseases lacking effective vaccines).

Some aspects of the present disclosure provide antigen nanoparticles and immunogenic compositions (e.g., vaccine compositions) thereof comprising a Beta coronavirus antigen which is associated with a nanoparticle. Some aspects of the present disclosure provide immunogenic compositions (e.g., vaccine compositions) comprising such an antigen nanoparticle and an adjuvantation system. In some embodiments, the Beta coronavirus antigen is a Beta coronavirus protein antigen or a fragment thereof. In some embodiments, the Beta coronavirus antigen is a Beta coronavirus spike protein receptor binding domain (RBD). In some embodiments, the nanoparticle comprises a multimeric protein scaffold comprising subunits of Aquifex aeolicus lumazine synthase (LuS). In some embodiments, the Beta coronavirus antigen (e.g., spike protein RBD) is covalently attached to subunits of the multimeric protein scaffold (e.g., LuS). In some embodiments, immunogenic compositions (e.g., vaccine compositions) comprising the antigen nanoparticle comprise an adjuvantation system comprising a squalene-based oil-in-water emulsion (OIW). An immunogenic composition (e.g., a vaccine composition) provided herein may be used in methods of inducing an immune response to an antigen in a subject in need thereof, the method comprising administering to the subject an effective amount of a Beta coronavirus antigen nanoparticle and an effective amount of the adjuvantation system (e.g., an effective amount of a nanoparticle comprising Beta coronavirus spike protein receptor binding domain (RBD-NP) and an effective amount of an OIW). In some embodiments, the immunogenic composition (e.g., vaccine composition) described herein may be used for inducing an immune response in a subject that is a newborn, an adult, or an elderly subject (e.g., a human subject older than 65 years old). In particular, the immunogenic composition (e.g., vaccine composition) described herein is effective for elderly immunization (i.e., for immunizing a human subject older than 65 years old).

“Beta coronavirus” is one of four genera (Alpha-, Beta-, Gamma-, and Delta-) of coronaviruses. It is in the subfamily Orthocoronavirinae in the family Coronaviridae, of the order Nidovirales. They are enveloped, positive-sense, single-stranded RNA viruses of zoonotic origin. Beta coronaviruses of the greatest clinical importance concerning humans SARS-CoV-1 (which causes severe acute respiratory syndrome, also referred to as SARS) and SARS-CoV-2 (which causes coronavirus disease 2019, also referred to as COVID-19), and MERS-CoV (which causes Middle East respiratory syndrome, also referred to as MERS).

An “antigen” refers to an entity that is bound by an antibody or receptor, or an entity that induces the production of the antibody. In some embodiments, an antigen increases the production of antibodies that specifically bind the antigen. In some embodiments, an antigen comprises a protein or polypeptide. Such protein or peptide are referred to herein as “immunogenic polypeptide.” For the purpose of the present disclosure, the antigen may comprise parts (e.g., viral coat proteins) of a Beta coronavirus (e.g., MERS-CoV, SARS-CoV-1, or SARS-CoV-2).

In some embodiments, a protein or polypeptide antigen is a wild type (“native”) protein or polypeptide. In some embodiments, a protein or polypeptide antigen is a polypeptide variant to a wild type protein or polypeptide. The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, polypeptide variants possess at least 50% identity to a native or reference sequence. In some embodiments, variants share at least 70%, at least 80%, at least 90%, at least 95%, or at least 99% identity with a native or reference sequence.

The term “nanoparticle” as used within the present disclosure refers to a solid, semi-solid, or liquid composition (“matrix”) having a mean particle size that is at least 10 nanometers but less than 1000 nanometers in diameter across its longest axis. In some embodiments, a nanoparticle generally has a diameter between 10 nm and 100 nm across its longest axis. A nanoparticle may be composed of naturally occurring, partially synthetic (i.e., not naturally occurring), or entirely synthetic materials. A nanoparticle may be heterogenous, being evenly composed of the same materials throughout, or may be heterogenous in composition. A nanoparticle may be approximately spherical in shape, or may be approximately ellipsoidal or cylindrical in shape. A nanoparticle may or may not be hollow, as defined by having a core at approximately its geometric center which is distinct in composition from that of the rest of the nanoparticle. A hollow nanoparticle may be void at its center, or may comprise a solid, semi-solid, liquid, or gas that is chemically distinct from the matrix. A collection of nanoparticles having the same composition may be approximately homogenous in size, i.e., having approximately the same diameter across their longest axis, or may be heterogenous in size, i.e., having variable diameters across their longest axis.

In some embodiments, a nanoparticle comprises a protein scaffold that comprises multiple protein subunits. In some embodiments, the protein subunits of the scaffold spontaneously assemble into the nanoparticle. Various examples of self-assembling protein nanoparticles (SAPNs) are known in the art and include certain viral proteins (e.g., hemagglutinin, human papilloma virus L1 major capsid protein, Hepatitis B surface antigen, bacteriophage Qβ), as well as certain bacterial proteins (e.g., ferritin, encapsuling, lumazine synthase) (see, e.g., López-Sagaseta J, et al. “Self-assembling protein nanoparticles in the design of vaccines.” Comput Struct Biotechnol J. 2015 Nov. 26; 14:58-68, which is incorporated by reference herein).

In some embodiments, a nanoparticle described in the present disclosure comprises a protein scaffold comprising multiple (e.g., approximately 60 or more) subunits of Aquifex aeolicus lumazine synthase (also known as 6,7-dimethyl-8-ribityllumazine synthase or DMRL synthase, hereafter abbreviated as “LuS”). In some embodiments, LuS subunits spontaneously assemble into approximately icosahedral nanoparticles with a mean diameter of 50 nm. In some embodiments, a nanoparticle comprising approximately 60 subunits of LuS is characterized as having T=1 icosahedral symmetry, according to well established conventions for identifying the shape and size of viral and virus-like particles which are known in the art (see, e.g., Prasad B V & Schmid, M F, “Principles of virus structural organization.” Viral Molecular Machines. 2012; 726: 17-47, which is incorporated by reference herein). In some embodiments, a protein scaffold that comprises multiple LuS subunits may assemble into approximately icosahedral nanoparticles with a mean diameter larger than 50 nm. In such embodiments, a nanoparticle comprising more than 60 subunits of LuS may be characterized as having, for example, T=3, T=5, or T=7 icosahedral symmetry.

In some embodiments, a nanoparticle comprising a protein scaffold comprising multiple LuS subunits having the following amino acid sequence, which is an example amino acid sequence from A. aeolicus (UniProtKB/Swiss-Prot: O66529.1):

(SEQ ID NO: 1)
MQIYEGKLTAEGLRFGIVASRFNHALVDRLVEGAIDCIVRHGGREEDIT
LVRVPGSWEIPVAAGELARKEDIDAVIAIGVLIRGATPHFDYIASEVSK
GLANLSLELRKPITFGVITADTLEQAIERAGTKHGNKGWEAALSAIEMA
NLFKSLR.

In some embodiments, a nanoparticle comprising a protein scaffold comprising multiple LuS subunits may be produced by recombinantly expressing the following genetic sequence, which is an example A. aeolicus gene encoding LuS (Gene ID 1192672):

(SEQ ID NO: 2)
ATGCAAATTTACGAAGGGAAACTAACCGCTGAAGGGCTGAGGTTCGGTA
TAGTGGCTTCCAGGTTCAACCACGCACTCGTGGATAGACTAGTTGAGGG
AGCTATAGACTGCATAGTAAGACACGGGGGAAGGGAAGAAGACATAACG
CTCGTTAGAGTGCCGGGCTCCTGGGAAATTCCCGTGGCTGCGGGAGAGC
TTGCGAGAAAAGAGGACATAGACGCTGTGATAGCGATAGGAGTTCTAAT
AAGGGGGGCTACTCCCCACTTTGATTACATAGCCTCTGAAGTGTCAAAA
GGGCTTGCGAACCTTTCCTTAGAACTGAGAAAACCCATAACCTTCGGTG
TTATAACTGCGGACACCTTGGAGCAGGCGATAGAAAGGGCGGGAACAAA
GCACGGGAATAAGGGCTGGGAAGCTGCACTTTCCGCAATAGAAATGGCA
AACTTATTTAAGAGTCTGAGATGA.

In some embodiments, a protein subunit (e.g., LuS) for assembling a nanoparticle comprising a protein scaffold is modified relative to its wild-type (“native”) amino acid sequence. In some embodiments, a protein subunit (e.g., LuS) is a variant protein subunit comprising amino acid substitutions, insertions, and/or deletions in relation to a reference sequence (e.g., that of a wild-type protein subunit). In some embodiments, a protein subunit (e.g., LuS) is modified by insertion of a sequence tag to the original amino acid sequence. A tag may be used to aid in the enrichment of the protein subunit when expressed and collected recombinantly. A tag may also be used to facilitate an association (interaction) between the protein subunit (e.g., LuS) and a second protein. Such an interaction may be covalent or non-covalent. A covalent association between a protein subunit of a nanoparticle and a second protein may also be referred to herein as being “conjugated to” the second protein.

The term “antigen nanoparticle” as used within the present disclosure refers to a nanoparticle that comprises one or more antigens derived from a pathogen (e.g., one or more protein antigens). In some embodiments, an antigen nanoparticle comprises a nanoparticle comprising protein subunits that are associated with (stably interacting with) one or more antigens derived from a pathogen (e.g., one or more protein antigens). In some embodiments, subunits of the nanoparticle and the one or more antigens derived from a pathogen associate (interact) covalently. In some embodiments, subunits of the nanoparticle and the one or more antigens derived from a pathogen associate (interact) non-covalently. In some embodiments where an antigen nanoparticle comprises a nanoparticle comprising protein subunits that are associated with one or more antigens derived from a pathogen, the nanoparticle may be said to “display” the one or more antigens. In some embodiments, the one or more antigens are displayed on the surface of the nanoparticle. In embodiments where the nanoparticle is hollow, and thus possesses both an internal and external surface, the one or more antigens may be displayed on the external surface of the nanoparticle such that antigens may be readily recognized by the immune system of a subject. In some embodiments, the one or more antigens from a pathogen which are associated with a nanoparticle are protein antigens of a Beta coronavirus (e.g., MERS-CoV, SARS-CoV-1, or SARS-CoV-2).

In some embodiments, the protein subunits of an antigen nanoparticle (e.g., LuS) and/or one or more antigens derived from a pathogen (e.g., a MERS-CoV, SARS-CoV-1, or SARS-CoV-2 protein antigen) are modified by one or more tags to facilitate association. In some embodiments, either of the subunits protein subunits of an antigen nanoparticle (e.g., LuS) or the one or more antigens derived from a pathogen (e.g., a MERS-CoV, SARS-CoV-1, or SARS-CoV-2 protein antigen) are modified by SpyCatcher and the other is modified by SpyTag, as are well known in the art (see, e.g., Zakeri, B et al. “Peptide tag forming a rapid covalent bond to a protein, through engineering a bacterial adhesin.” Proc. Natl Acad. Sci. USA 2012; 109, E690-E697; and Hatlem D et al. “Catching a SPY: Using the SpyCatcher-SpyTag and Related Systems for Labeling and Localizing Bacterial Proteins.” Int J Mol Sci. 2019; April 30; 20(9):2129, which are incorporated herein by reference). In such embodiments, a covalent interaction (an isopeptide bond) occurs spontaneously when SpyCatcher and SpyTag contact one another, thereby resulting in association between protein subunits of the nanoparticle and the one or more antigens, each modified with SpyCatcher or SpyTag. In some embodiments, protein subunits of the antigen nanoparticle (e.g., LuS) are modified with SpyCatcher, while the one or more antigens derived from a pathogen (e.g., a MERS-CoV, SARS-CoV-1, or SARS-CoV-2 protein antigen) are modified with SpyTag. In some embodiments, protein subunits of the antigen nanoparticle (e.g., LuS) are modified with SpyTag, while the one or more antigens derived from a pathogen (e.g., a MERS-CoV, SARS-CoV-1, or SARS-CoV-2 protein antigen) are modified with SpyCatcher. Association between modified (tagged) protein subunits of the antigen nanoparticle and modified (tagged) antigens derived from a pathogen may occur prior to or following assembly of the nanoparticle. The antigen nanoparticle may be produced, for example, by contacting pre-assembled nanoparticles (e.g., nanoparticles comprising a protein scaffold comprising modified (tagged) LuS) with one or more antigens derived from a pathogen (e.g., a modified (tagged) MERS-CoV, SARS-CoV-1, or SARS-CoV-2 protein antigen).

In some embodiments, an antigen nanoparticle comprising a protein scaffold and one or more protein antigens derived from a pathogen, comprise either a subunit protein of the scaffold or an antigen protein which is modified with the following amino acid sequence, which corresponds to SpyCatcher (GenBank: AFD50637.1):

(SEQ ID NO: 3)
MSYYHHHHHHDYDIPTTENLYFQGAMVDTLSGLSSEQGQSGDMTIEEDS
ATHIKFSKRDEDGKELAGATMELRDSSGKTISTWISDGQVKDFYLYPGK
YTFVETAAPDGYEVATAITFTVNEQGQVTVNGKATKGDAHI.

In some embodiments, an antigen nanoparticle comprising a protein scaffold and one or more protein antigens derived from a pathogen, comprise either a subunit protein of the scaffold or an antigen protein which is modified with the following amino acid sequence, which corresponds to SpyTag (PDB: 4MLS_B): AHIVMVDAYKPTK (SEQ ID NO: 4).

Antigen nanoparticles (e.g., nanoparticles comprising LuS and one or more MERS-CoV, SARS-CoV-1, or SARS-CoV-2 protein antigens) may be produced by modifying protein subunits of the antigen nanoparticle (e.g., LuS) and one or more antigens derived from a pathogen (e.g., a MERS-CoV, SARS-CoV-1, or SARS-CoV-2 protein antigen) with one or more alternative tags which are known in the art. Alternative tags for facilitating covalent association between the nanoparticle subunits and protein antigen(s) include derivatives of SpyCatcher and SpyTag, such as but not limited to SpyCatcher002 and SpyTag002 (see, e.g., Keeble, A H et al., “Evolving accelerated amidation by SpyTag/SpyCatcher to analyze membrane dynamics”. Angew. Chem. Int. Ed., 2017; 56, 16521-16525, which is incorporated by reference herein). Alternative tags for facilitating non-covalent association between the nanoparticle subunits and protein antigen(s) include a wide variety of tags for facilitating protein-protein interaction as well known in the art, including but not limited to albumin and albumin binding protein, biotin-carboxy carrier protein and avidin, calmodulin and calmodulin binding protein, choline-binding domain and choline-binding domain peptide, and streptavidin-binding peptide and streptavidin, as well as monoclonal antibody fragments and any peptide that the monoclonal antibody fragment is specific for, such as but not limited to alkaline phosphatase, bacteriophage T7 epitope, Bluetongue virus tag, E2 epitope, FLAG epitope, human influenza hemagglutinin, HSV epitope, KT3 epitope, or Myc epitope (see, e.g., Kimple, M E et al, “Overview of affinity tags for protein purification.” Current protocols in protein science, 2013; 73, 9.9.1-9.9.23, which is incorporated by reference herein).

An “adjuvantation system” refers to a composition comprising one or more adjuvants. An “adjuvant” refers to a pharmacological or immunological agent that modifies the effect of other agents, for example, of an antigen in a vaccine. Adjuvants are typically included in vaccines to enhance the recipient subject's immune response to an antigen. The use of adjuvants allows the induction of a greater immune response in a subject with the same dose of antigen, or the induction of a similar level of immune response with a lower dose of injected antigen. Adjuvants are thought to function in several ways, including by increasing the surface area of antigen, prolonging the retention of the antigen in the body thus allowing time for the lymphoid system to have access to the antigen, slowing the release of antigen, targeting antigen to macrophages, activating macrophages, activating leukocytes such as antigen-presenting cells (e.g., monocytes, macrophages, and/or dendritic cells), or otherwise eliciting broad activation of the cells of the immune system see, e.g., H. S. Warren et al, Annu. Rev. Immunol., 4:369 (1986), incorporated herein by reference. The ability of an adjuvant to induce and increase a specific type of immune response and the identification of that ability is thus a key factor in the selection of particular adjuvants for vaccine use against a particular pathogen. Adjuvants that are known to those of skill in the art, include, without limitation: aluminum salts (referred to herein as “alum”), liposomes, lipopolysaccharide (LPS) or derivatives such as monophosphoryl lipid A (MPLA) and glycopyranosyl lipid A (GLA), molecular cages for antigen, components of bacterial cell walls, endocytosed nucleic acids such as double-stranded RNA (dsRNA), single-stranded DNA (ssDNA), and unmethylated CpG dinucleotide-containing DNA (CpG-ODN). Typical adjuvants include water and oil emulsions, e.g., Freund's adjuvant and MF59, and chemical compounds such as aluminum hydroxide or alum. At present, currently licensed vaccines in the United States contain only a limited number of adjuvants, such as alum that enhances production of TH 2 cells and MPLA which activates innate immunity via Toll-like receptor 4 (TLR4). Many of the most effective adjuvants include bacteria or their products, e.g., microorganisms such as the attenuated strain of Mycobacterium bovis, Bacille Calmette-Guérin (BCG); microorganism components, e.g., alum-precipitated diphtheria toxoid, bacterial lipopolysaccharides (“endotoxins”) and their derivatives such as MPLA and GLA.

In some embodiments, the adjuvantation system of the present disclosure comprises a squalene-based oil-in-water emulsion (OIW). Various examples of OIWs are known to those of skill in the art and include, without limitation, formulations that are commercially available as AddaVax and AddaS03 (AS03). OIWs are characterized by several key characteristics, including being primarily composed of squalene oil and filtered to a particular particle size. For example, AddaVax is a nanoscale emulsion comprising two components: sorbitan trioleate (0.5% w/v) in squalene oil (5% v/v), and polysorbate 80 (Tween 80; 0.5% w/v) in sodium citrate buffer (10 mM, pH 6.5), which is produced through a microfluidizer and filter sterilized to a maximum 0.22 μm in size, thereby generating particles that are on average 160 nm in diameter. Other OIW formulations are similar, though composed of distinct formulations. For example, AddaS03 are composed of DL-α-tocopherol (Vitamin E), squalene, and polysorbate 80. Without wishing to be bound by theory, the mechanism by which OIWs enhance immunity toward antigens remains incompletely understood, however OIWs have been shown to elicit NF-κB-dependent innate immune responses at both the site of administration (e.g. intramuscular (i.m.) injection) and in draining lymph nodes of immunized subjects, thereby enhancing expression of cytokines and chemokines that upregulate production of antigen-specific antibodies belonging to certain immunoglobulin subtypes, while also enhancing the response of CD8+ T cells (see, e.g., Morel S, et al “Adjuvant System AS03 containing α-tocopherol modulates innate immune response and leads to improved adaptive immunity.” Vaccine, 2011; 29(13):2461-73; Garcon N, et al., “Development and evaluation of AS03, an Adjuvant System containing α-tocopherol and squalene in an oil-in-water emulsion.” Expert Rev Vaccines. 2012; 11(3):349-66; Fochesato M et al. “Comparative preclinical evaluation of AS01 versus other Adjuvant Systems in a candidate herpes zoster glycoprotein E subunit vaccine.” Hum Vaccin Immunother, 2016; 12(8): 2092-2095; and Kim E H, et al. “Squalene emulsion-based vaccine adjuvants stimulate CD8 T cell, but not antibody responses, through a RIPK3-dependent pathway.” Elife, 2020; 9:e52687, which are incorporated by reference herein). Related adjuvants include liposomal adjuvants, which are produced by similar means and composed of similar materials with the exception of squalene. For example, the commercially available liposomal adjuvant AS01B is composed of 3-O-desacyl-4′-monophosphoryl lipid A (MPL) from Salmonella minnesota and a saponin molecule (QS-21) purified from plant extract Quillaja saponaria Molina, in an oil formulation comprising dioleoyl phosphatidylcholine (DOPC) and cholesterol. Despite the established use of OIWs and related oil-based adjuvants in immunization against other pathogens, including human influenza virus and varicella-zoster (Shingles) virus, many OIWs have not yet been evaluated in the context of Beta coronavirus protein antigens, especially SARS-CoV-2 protein antigens and immunogenic fragments thereof.

In some embodiments, an adjuvantation system comprising an OIW further comprises a second adjuvant. A second adjuvant may be any adjuvant that is known to those of skill in the art, including, without limitation: alum, liposomes, LPS or derivatives such as MPLA and GLA, molecular cages for antigen, components of bacterial cell walls, endocytosed nucleic acids such as dsRNA, ssDNA, and unmethylated CpG dinucleotide-containing DNA, and water and oil emulsions such as Freund's adjuvant and MF59. A second adjuvant may be an agonist of one or more pattern recognition receptors (PRRs), including one or more Toll-like receptors (TLRs) that recognize pathogen-associated molecular patterns (PAMPs) produced by infectious microorganisms, thereby signaling for the production of cytokines that regulate immune activation, inflammation, survival, and proliferation. An “agonist” is a chemical that binds to a receptor and activates the receptor to produce a biological response. A second adjuvant may be an agonist of one or more TLRs located on the cell surface (e.g., TLR1, TLR2, TLR4, TLR5, TLR6, and TLR11) or the lysosomal and/or endosomal surface (e.g., TLR1, TLR2, TLR4, TLR5, TLR6, and TLR11) of dendritic cells, macrophages, natural killer cells, T cells, B cells, and/or non-immune cells. TLR agonists known in the art include, for example, LPS and MPLA (TLR4 agonists), bacterial flagellar proteins (TLR5 agonists), resiquimod (TLR7/8 agonist), and CpG-ODNs (TLR9 agonists). Additional examples of TLR agonists known in the art have been described, e.g., by Mifsud et al., Front Immunol. 2014 Mar. 3; 5:79; Owen et al., Front Immunol. 2021 Feb. 18; 11:622614; and Dowling et al., ImmunoHorizons, 2018 Jul. 1, 2(6):185-197, which are incorporated herein by reference in their entirety.

Adjuvants or adjuvantation systems are used in immunogenic compositions (e.g., the Beta coronavirus immunogenic composition (e.g., vaccine composition)) described herein. The terms “vaccine composition” and “vaccine” are used interchangeably herein. An “immunogenic composition” is a composition that activates or enhances a subject's immune response to an antigen after the vaccine is administered to the subject. Vaccine compositions are a type of immunogenic compositions. In some embodiments, an immunogenic composition stimulates the subject's immune system to recognize the antigen (e.g., a Beta coronavirus antigen) as foreign, and enhances the subject's immune response if the subject is later exposed to the pathogen (e.g., Beta coronavirus), whether attenuated, inactivated, killed, or not. Vaccines may be prophylactic, for example, preventing or ameliorating a detrimental effect of a future exposure to a pathogen (e.g., Beta coronavirus), or therapeutic, for example, activating the subject's immune response to a pathogen after the subject has been exposed to the pathogen (e.g., Beta coronavirus). In some embodiments, an immunogenic composition (e.g., vaccine composition) is used to protect or treat an organism against a disease (e.g., MERS, SARS and/or COVID-19). In some embodiments, an immunogenic composition is contemplated which comprises an antigen derived from a Beta coronavirus (e.g., MERS-CoV, SARS-CoV-1, or SARS-CoV-2) and an OIW or liposomal adjuvant.

In some embodiments, the vaccine is a subunit vaccine (e.g., a recombinant subunit Beta coronavirus (e.g., MERS-CoV, SARS-CoV-1, or SARS-CoV-2) vaccine in which the vaccine comprises a protein antigen (e.g., a MERS-CoV, SARS-CoV-1, or SARS-CoV-2 protein antigen). In some embodiments, the protein antigen is a whole (i.e., full length) protein or a fragment thereof (e.g., a protein domain). In some embodiments, the protein antigen is covalently attached to another protein, such as that of a nanoparticle comprising protein subunits (e.g., a nanoparticle comprising subunits of lumazine synthase).

In some embodiments, a polypeptide variant comprises substitutions, insertions, deletions. In some embodiments, a polypeptide variant encompasses covalent variants and derivatives. The term “derivative” is used synonymously with the term “variant” but generally refers to a molecule that has been modified and/or changed in any way relative to a reference molecule or starting molecule.

In some embodiments, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

In some embodiments, the polypeptide variants comprise at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule. In some embodiments, the antigen is a polypeptide that includes 2, 3, 4, 5, 6, 7, 8, 9, 10, or more substitutions compared to a reference protein.

In some embodiments, the substitution is a conservative amino acids substitution. The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In some embodiments, protein fragments, functional protein domains, and homologous proteins are used as antigens in accordance with the present disclosure. For example, an antigen may comprise any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to a reference protein (e.g., a protein from a microbial pathogen) herein can be utilized in accordance with the disclosure.

In some embodiments, the antigen comprises more than one immunogenic proteins or polypeptides (e.g., 2, 3, 4, 5, 6, 7, 8, 9, 10, or more). In some embodiments, the more than one immunogenic proteins or polypeptides are derived from one protein (e.g., different fragments or one protein). In some embodiments, the more than one immunogenic proteins or polypeptides are derived from multiple proteins (e.g., from 2, 3, 4, 5, 6, 7, 8, 9, 10, or more proteins).

In some embodiments, the antigen comprises one or more immunogenic proteins, protein fragments or polypeptides that share at least 50%, at least 60%, at least 70%, at least 80%, at least 90%, at least 95%, or at least 99% identity to a reference sequence of a particular Beta coronavirus variant. In some embodiments, the variant is wild-type MERS-CoV, SARS-CoV-1, or SARS-CoV-2. In some embodiments, the variant is a Beta coronavirus variant that is not a wild-type variant. In some embodiments, the variant is a variant of SARS-CoV-2 that is not wild-type SARS-CoV-2, such as, but not limited to, B.1.1.7, B.1.351, P.1, B.1.427, B.1.429, B.1.526, B.1.526.1, B.1.525, P.2, B.1.617, B.1.617.1, B.1.617.2, or B.1.617.3 SARS-CoV-2.

Proteins or polypeptides of the present disclosure may share a certain degree of sequence similarity or identity with reference molecules (e.g., reference polypeptides), for example, wild-type molecules. The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide sequences is defined as the percentage of residues (amino acid residues) in the candidate amino acid sequence that are identical with the residues in the amino acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that of a particular reference polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g., between nucleic acid molecules (e.g., DNA molecules and/or RNA molecules) and/or between polypeptide molecules. Polymeric molecules (e.g., nucleic acid molecules (e.g., DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original gene.

The term “identity” refers to the overall relatedness between polymeric molecules, for example, between polynucleotide molecules (e.g., DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In some embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

In some embodiments, the immunogenic compositions (e.g., vaccine compositions) described herein induce an immune response to a Beta coronavirus antigen (e.g., an antigen from any Beta coronavirus such as an antigen from MERS-CoV, SARS-CoV-1, or SARS-CoV-2) or to a Beta coronavirus (any Beta coronavirus species such as MERS-CoV, SARS-CoV-1, or SARS-CoV-2). In some embodiments, Beta coronavirus antigen used in the immunogenic composition described herein comprises a protein antigen from MERS-CoV. In some embodiments, Beta coronavirus antigen used in the immunogenic composition described herein comprises a protein antigen from SARS-CoV-1. In some embodiments, Beta coronavirus antigen used in the immunogenic composition described herein comprises a protein antigen from SARS-CoV-2. In some embodiments, the immunogenic composition (e.g., vaccine composition) induces an immune response against MERS-CoV, SARS-CoV-1 and/or SARS-CoV-2. Heterologous immunity is contemplated herein. Heterologous immunity refers to phenomenon by which antigen-specific response that were generated against one pathogen are reactivated in response to a second pathogen. For example, the immunogenic composition (e.g., vaccine composition) may comprises a SARS-CoV-1 antigen and induces immune response to both SARS-CoV-1 and SARS-CoV-2. Similarly, the immunogenic composition (e.g., vaccine composition) may comprises a SARS-CoV-2 antigen and induces immune response to both SARS-CoV-1 and SARS-CoV-2.

In some embodiments, the Beta coronavirus antigen used in the immunogenic composition (e.g., vaccine composition) described herein comprises a Beta coronavirus (e.g., MERS-CoV, SARS-CoV-1, or SARS-CoV-2) protein or polypeptide, or an immunogenic fragment or variant thereof. In some embodiments, the Beta coronavirus antigen is an immunogenic fragment that is a domain within a Beta coronavirus (e.g., MERS-CoV, SARS-CoV-1, or SARS-CoV-2) protein.

In some embodiments, the Beta coronavirus antigen in the immunogenic composition (e.g., vaccine composition) described herein comprises a MERS-CoV spike protein, or an immunogenic fragment thereof (e.g., the receptor binding domain of the spike protein). In some embodiments, the Beta coronavirus antigen in the immunogenic composition (e.g., vaccine composition) described herein comprises a SARS-CoV-1 spike protein. In some embodiments, the Beta coronavirus antigen in the immunogenic composition (e.g., vaccine composition) described herein comprises a SARS-CoV-2 spike protein, or an immunogenic fragment thereof (e.g., the receptor binding domain of the spike protein).

Amino acid sequences of examples of Beta coronavirus antigens in the immunogenic composition (e.g., vaccine composition) described herein are provided in Table 1.

TABLE 1
Beta coronavirus antigens

| Antigen | Amino Acid Sequence |
|---|---|
| SARS-CoV-1 | MFIFLLFLTLTSGSDLDRCTTFDDVQAPNYTQHTSSMRGVYYPDEIFRSDTLYLTQDLFLPFY |
| Spike Protein | SNVTGFHTINHTFGNPVIPFKDGIYFAATEKSNVVRGWVFGSTMNNKSQSVIIINNSTNVVIR |
| (SEQ ID NO: 5) | ACNFELCDNPFFAVSKPMGTQTHTMIFDNAFNCTFEYISDAFSLDVSEKSGNFKHLREFVFK |
| | NKDGFLYVYKGYQPIDVVRDLPSGFNTLKPIFKLPLGINITNFRAILTAFSPAQDIWGTSAAAY |
| | FVGYLKPTTFMLKYDENGTITDAVDCSQNPLAELKCSVKSFEIDKGIYQTSNFRVVPSGDVVR |
| | FPNITNLCPFGEVFNATKFPSVYAWERKKISNCVADYSVLYNSTFFSTFKCYGVSATKLNDLC |
| | FSNVYADSFVVKGDDVRQIAPGQTGVIADYNYKLPDDFMGCVLAWNTRNIDATSTGNYNYK |
| | YRYLRHGKLRPFERDISNVPFSPDGKPCTPPALNCYWPLNDYGFYTTTGIGYQPYRVVVLSF |
| | ELLNAPATVCGPKLSTDLIKNQCVNFNFNGLTGTGVLTPSSKRFQPFQQFGRDVSDFTDSVR |
| | DPKTSEILDISPCSFGGVSVITPGTNASSEVAVLYQDVNCTDVSTAIHADQLTPAWRIYSTGN |
| | NVFQTQAGCLIGAEHVDTSYECDIPIGAGICASYHTVSLLRSTSQKSIVAYTMSLGADSSIAYSN |
| | NTIAIPTNFSISITTEVMPVSMAKTSVDCNMYICGDSTECANLLLQYGSFCTQLNRALSGIAAE |
| | QDRNTREVFAQVKQMYKTPTLKYFGGFNFSQILPDPLKPTKRSFIEDLLFNKVTLADAGFM |
| | KQYGECLGDINARDLICAQKFNGLTVLPPLLTDDMIAAYTAALVSGTATAGWTFGAGAALQI |
| | PFAMQMAYRFNGIGVTQNVLYENQKQIANQFNKAISQIQESLTTTSTALGKLQDVVNQNAQ |
| | ALNTLVKQLSSNFGAISSVLNDILSRLDKVEAEVQIDRLITGRLQSLQTYVTQQLIRAAEIRAS |
| | ANLAATKMSECVLGQSKRVDFCGKGYHLMSFPQAAPHGVVFLHVTYVPSQERNFTTAPAIC |
| | HEGKAYFPREGVFVFNGTSWFITQRNFFSPQIITTDNTFVSGNCDVVIGIINNTVYDPLQPEL |
| | DSFKEELDKYFKNHTSPDVDLGDISGINASVVNIQKEIDRLNEVAKNLNESLIDLQELGKYEQ |
| | YIKWPWYVWLGFIAGLIAIVMVTILLCCMTSCCSCLKGACSCGSCCKFDEDDSEPVLKGVKLH |
| | YT |
| SARS-CoV-1 | RVVPSGDVVRFPNITNLCPFGEVFNATKFPSVYAWERKKISNCVADYSVLYNSTFFSTFKCYG |
| Spike Protein | VSATKLNDLCFSNVYADSFVVKGDDVRQIAPGQTGVIADYNYKLPDDFMGCVLAWNTRNID |
| receptor | ATSTGNYNYKYRYLRHGKLRPFERDISNVPFSPDGKPCTPPALNCYWPLNDYGFYTTTGIGY |
| binding | QPYRVVVLSFELLNAPATVCGPKLSTDLIKNQCVNF |
| domain (SEQ | |
| ID NO: 6) | |
| SARS-CoV-2 | MFVFLVLLPLVSSQCVNLTTRTQLPPAYTNSFTRGVYYPDKVFRSSVLHSTQDLFLPFFSNVT |
| Spike Protein | WFHAIHVSGTNGTKRFDNPVLPFNDGVYFASTEKSNIIRGWIFGTTLDSKTQSLLIVNNATN |
| (SEQ ID NO: 7) | VVIKVCEFQFCNDPFLGVYYHKNNKSWMESEFRVYSSANNCTFEYVSQPFLMDLEGKQGNF |
| | KNLREFVFKNIDGYFKIYSKHTPINLVRDLPQGFSALEPLVDLPIGINITRFQTLLALHRSYLT |
| | PGDSSSGWTAGAAAYYVGYLQPRTFLLKYNENGTITDAVDCALDPLSETKCTLKSFTVEKGI |
| | YQTSNFRVQPTESIVRFPNITNLCPFGEVFNATRFASVYAWNRKRISNCVADYSVLYNSASFS |
| | TFKCYGVSPTKLNDLCFTNVYADSFVIRGDEVRQIAPGQTGKIADYNYKLPDDFTGCVIAWN |
| | SNNLDSKVGGNYNYLYRLFRKSNLKPFERDISTEIYQAGSTPCNGVEGFNCYFPLQSYGFQPT |
| | NGVGYQPYRVVVLSFELLHAPATVCGPKKSTNLVKNKCVNFNFNGLTGTGVLTESNKKFLPF |
| | QQFGRDIADTTDAVRDPQTLEILDITPCSFGGVSVITPGTNTSNQVAVLYQDVNCTEVPVAIH |
| | ADQLTPTWRVYSTGSNVFQTRAGCLIGAEHVNNSYECDIPIGAGICASYQTQTNSPRRARSV |
| | ASQSIIAYTMSLGAENSVAYSNNSIAIPTNFTISVTTEILPVSMTKTSVDCTMYICGDSTECSNL |
| | LLQYGSFCTQLNRALTGIAVEQDKNTQEVFAQVKQIYKTPPIKDFGGFNFSQILPDPSKPSKR |
| | SFIEDLLFNKVTLADAGFIKQYGDCLGDIAARDLICAQKFNGLTVLPPLLTDEMIAQYTSALL |
| | AGTITSGWTFGAGAALQIPFAMQMAYRFNGIGVTQNVLYENQKLIANQFNSAIGKIQDSLSS |
| | TASALGKLQDVVNQNAQALNTLVKQLSSNFGAISSVLNDILSRLDKVEAEVQIDRLITGRLQS |
| | LQTYVTQQLIRAAEIRASANLAATKMSECVLGQSKRVDFCGKGYHLMSFPQSAPHGVVFLHV |
| | TYVPAQEKNFTTAPAICHDGKAHFPREGVFVSNGTHWFVTQRNFYEPQIITTDNTFVSGNC |
| | DVVIGIVNNTVYDPLQPELDSFKEELDKYFKNHTSPDVDLGDISGINASVVNIQKEIDRLNEV |
| | AKNLNESLIDLQELGKYEQYIKWPWYIWLGFIAGLIAIVMVTIMLCCMTSCCSCLKGCCSCGS |
| | CCKFDEDDSEPVLKGVKLHYT |
| SARS-CoV-2 | RVQPTESIVRFPNITNLCPFGEVFNATRFASVYAWNRKRISNCVADYSVLYNSASFSTFKCYG |
| Spike Protein | VSPTKLNDLCFTNVYADSFVIRGDEVRQIAPGQTGKIADYNYKLPDDFTGCVIAWNSNNLDS |
| receptor | KVGGNYNYLYRLFRKSNLKPFERDISTEIYQAGSTPCNGVEGFNCYFPLQSYGFQPTNGVGYQ |
| binding | PYRVVVLSFELLHAPATVCGPKKSTNLVKNKCVNF |
| domain (SEQ | |
| ID NO: 8) | |
| MERS Spike | MIHSVFLLMFLLTPTESYVDVGPDSVKSACIEVDIQQTFFDKTWPRPIDVSKADGIIYPQGRT |
| protein (SEQ | YSNITITYQGLFPYQGDHGDMYVYSAGHATGTTPQKLFVANYSQDVKQFANGFVVRIGAAA |
| ID NO: 9) | NSTGTVIISPSTSATIRKIYPAFMLGSSVGNFSDGKMGRFFNHTLVLLPDGCGTLLRAFYCILE |
| | PRSGNHCPAGNSYTSFATYHTPATDCSDGNYNRNASLNSFKEYFNLRNCTFMYTYNITEDEI |
| | LEWFGITQTAQGVHLFSSRYVDLYGGNMFQFATLPVYDTIKYYSIIPHSIRSIQSDRKAWAAF |
| | YVYKLQPLTFLLDFSVDGYIRRAIDCGFNDLSQLHCSYESFDVESGVYSVSSFEAKPSGSVVEQ |
| | AEGVECDFSPLLSGTPPQVYNFKRLVFTNCNYNLTKLLSLFSVNDFTCSQISPAAIASNCYSSL |
| | ILDYFSYPLSMKSDLSVSSAGPISQFNYKQSFSNPTCLILATVPHNLTTITKPLKYSYINKCSRL |
| | LSDDRTEVPQLVNANQYSPCVSTVPSTVWEDGDYYRKQLSPLEGGGWLVASGSTVAMTEQL |
| | QMGFGITVQYGTDTNSVCPKLEFANDTKIASQLGNCVEYSLYGVSGRGVFQNCTAVGVRQQR |
| | FVYDAYQNLVGYYSDDGNYYCLRACVSVPVSVIYDKETKTHATLFGSVACEHISSTMSQYSRS |
| | TRSMLKRRDSTYGPLQTPVGCVLGLVNSSLFVEDCKLPLGQSLCALPDTPSTLTPRSVRSVPG |
| | EMRLASIAFNHPIQVDQLNSSYFKLSIPTNFSFGVTQEYIQTTIQKVTVDCKQYVCNGFQKCE |
| | QLLREYGQFCSKINQALHGANLRQDDSVRNLFASVKSSQSSPIIPGFGGDFNLTLLEPVSISTG |
| | SRSARSAIEDLLFDKVTIADPGYMQGYDDCMQQGPASARDLICAQYVAGYKVLPPLMDVNM |
| | EAAYTSSLLGSIAGVGWTAGLSSFAAIPFAQSIFYRLNGVGITQQVLSENQKLIANKFNQALGA |
| | MQTGFTTTNEAFRKVQDAVNNNAQALSKLASELSNTFGAISASIGDIIQRLDVLEQDAQIDRL |
| | INGRLTTLNAFVAQQLVRSESAALSAQLAKDKVNECVKAQSKRSGFCGQGTHIVSFVVNAPN |
| | GLYFMHVGYYPSNHIEVVSAYGLCDAANPTNCIAPVNGYFIKTNNTRIVDEWSYTGSSFYAP |
| | EPITSLNTKYVAPQVTYQNISTNLPPPLLGNSTGIDFQDELDEFFKNVSTSIPNFGSLTQINTT |
| | LLDLTYEMLSLQQVVKALNESYIDLKELGNYTYYNKWPWYIWLGFIAGLVALALCVFFILCC |
| | TGCGTNCMGKLKCNRCCDRYEEYDLEPHKVHVH |
| MERS Spike | ECDFSPLLSGTPPQVYNFKRLVFTNCNYNLTKLLSLFSVNDFTCSQISPAAIASNCYSSLILDYF |
| protein | SYPLSMKSDLSVSSAGPISQFNYKQSFSNPTCLILATVPHNLTTITKPLKYSYINKC |
| receptor | |
| binding | |
| domain (SEQ | |
| ID NO: 10) | |

In some embodiments, the Beta coronavirus antigen in the immunogenic composition (e.g., vaccine composition) described herein comprises a protein having an amino acid sequence that is at least 70% (e.g., at least 70%, at least 75%, at least 80%, at least 85%, at least 90%, at least 95%, or at least 99%) identical to any one of SEQ ID NOs: 5-10. In some embodiments, the Beta coronavirus antigen in the immunogenic composition (e.g., vaccine composition) described herein comprises a protein having an amino acid sequence that is 70%, 75%, 80%, 85%, 90%, 95%, or 99% identical to any one of SEQ ID NOs: 5-10. In some embodiments, the Beta coronavirus antigen in the immunogenic composition (e.g., vaccine composition) described herein comprises a protein comprising the amino acid sequence of any one of SEQ ID NO: 5-10.

In some embodiments, the Beta coronavirus antigen in the immunogenic composition (e.g., vaccine composition) described herein is multimer, such as but not limited to a dimer or a trimer. In some embodiments, multimerization enhances the immunogenicity of the Beta coronavirus antigen when administered to a subject.

In some embodiments, the Beta coronavirus antigen in the immunogenic composition (e.g., vaccine composition) described herein is a component of an antigen nanoparticle. In some embodiments, the Beta coronavirus antigen is a Beta coronavirus (e.g., MERS-CoV, SARS-CoV-1, or SARS-CoV-2) spike protein or an immunogenic fragment thereof (e.g., the receptor binding domain of the spike protein) that is a component of an antigen nanoparticle. In some embodiments, the Beta coronavirus (e.g., MERS-CoV, SARS-CoV-1, or SARS-CoV-2) spike protein or an immunogenic fragment thereof (e.g., the receptor binding domain of the spike protein) associates (stably interacts) with protein subunits (e.g., LuS) of the antigen nanoparticle covalently. In some embodiments, the Beta coronavirus (e.g., MERS-CoV, SARS-CoV-1, or SARS-CoV-2) spike protein or an immunogenic fragment thereof (e.g., the receptor binding domain of the spike protein) associates (stably interacts) with protein subunits (e.g., LuS) of the antigen nanoparticle non-covalently. In some embodiments where the association (interaction) is covalent, either of the nanoparticle subunits (e.g., LuS) or the Beta coronavirus (e.g., MERS-CoV, SARS-CoV-1, or SARS-CoV-2) spike protein or an immunogenic fragment thereof (e.g., the receptor binding domain of the spike protein) is modified by SpyCatcher (SEQ ID NO: 3), or a derivative thereof, and the other is modified by SpyTag (SEQ ID NO: 4), or a derivative thereof. In some embodiments, an antigen nanoparticle contemplated herein is an antigen nanoparticle comprising a nanoparticle comprising a LuS protein scaffold and a Beta coronavirus (e.g., MERS-CoV, SARS-CoV-1, or SARS-CoV-2) receptor binding domain (RBD-NP). In some embodiments, association with a nanoparticle (i.e., as part of an antigen nanoparticle) enhances the immunogenicity of the Beta coronavirus antigen (e.g., MERS-CoV, SARS-CoV-1, or SARS-CoV-2 spike protein or spike protein receptor binding domain) when administered to a subject.

In some embodiments, the immunogenic composition (e.g., vaccine composition) described herein are formulated for administration to a subject. In some embodiments, the immunogenic composition (e.g., vaccine composition) is formulated or administered in combination with one or more pharmaceutically acceptable excipients. In some embodiments, immunogenic compositions (e.g., vaccine composition) comprise at least one additional active substance, such as, for example, a therapeutically active substance, a prophylactically active substance, or a combination of both. Immunogenic compositions (e.g., vaccine composition) may be sterile, pyrogen-free or both sterile and pyrogen-free. General considerations in the formulation and/or manufacture of pharmaceutical agents, such as immunogenic compositions (e.g., vaccine composition), may be found, for example, in Remington: The Science and Practice of Pharmacy 21st ed., Lippincott Williams & Wilkins, 2005 (incorporated herein by reference in its entirety).

Formulations of the immunogenic composition (e.g., vaccine composition) described herein may be prepared by any method known or hereafter developed in the art of pharmacology. In general, such preparatory methods include the step of bringing the antigen and/or the adjuvant (e.g., an antigen nanoparticle, such as a nanoparticle comprising Beta coronavirus spike protein receptor binding domain (RBD-NP), and an OIW) into association with an excipient and/or one or more other accessory ingredients, and then, if necessary and/or desirable, dividing, shaping and/or packaging the product into a desired single- or multi-dose unit.

Relative amounts of the antigen (e.g., an antigen nanoparticle), the adjuvant, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, the immunogenic composition (e.g., vaccine composition) described herein are formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA vaccines (e.g., for transplantation into a subject), hyaluronidase, nanoparticle mimics and combinations thereof.

In some embodiments, the immunogenic composition (e.g., vaccine composition) is formulated in an aqueous solution. In some embodiments, the immunogenic composition (e.g., vaccine composition) is formulated in a nanoparticle. In some embodiments, the immunogenic composition (e.g., vaccine composition) is formulated in a lipid nanoparticle. In some embodiments, the immunogenic composition (e.g., vaccine composition) is formulated in a lipid-polycation complex, referred to as a lipid nanoparticle. The formation of the lipid nanoparticle may be accomplished by methods known in the art and/or as described in U.S. Pub. No. 20120178702, incorporated herein by reference. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is incorporated herein by reference. In some embodiments, the immunogenic composition (e.g., vaccine composition) is formulated in a lipid nanoparticle that includes a non-cationic lipid such as, but not limited to, cholesterol or dioleoyl phosphatidylethanolamine (DOPE).

In some embodiments, a vaccine formulation described herein is a nanoparticle that comprises at least one lipid (termed a “lipid nanoparticle” or “LNP”). The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, incorporated herein by reference. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety). In some embodiments, the immunogenic composition (e.g., vaccine composition) described herein may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles may have a diameter from about 10 to 500 nm. In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm. In some embodiments, the immunogenic composition (e.g., vaccine composition) is formulated in a liposome. Liposomes are artificially prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are incorporated herein by reference.

In some embodiments, the immunogenic composition (e.g., vaccine composition) described herein may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)[1,3]-dioxolane (DLin-KC2-DMA), and MC3 (US20100324120; incorporated herein by reference) and liposomes which may deliver small molecule drugs such as, but not limited to, DOXIL® from Janssen Biotech, Inc. (Horsham, PA).

In some embodiments, the antigen and/or the adjuvantation system may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are incorporated herein by reference.

The antigen (e.g., an antigen nanoparticle), the adjuvantation system, and/or optionally the second adjuvant may be formulated using any of the methods described herein or known in the art separately or together. For example, the antigen and the adjuvantation system may be formulated in one lipid nanoparticle or two separately lipid nanoparticles. In some embodiments, the antigen, the adjuvantation system are formulated in the same aqueous solution or two separate aqueous solutions.

Other aspects of the present disclosure provide methods of inducing an immune response to Beta coronavirus (e.g., MERS-CoV, SARS-CoV-1, or SARS-CoV-2) or a Beta coronavirus (e.g., MERS-CoV, SARS-CoV-1, or SARS-CoV-2) antigen in a subject in need thereof, the method comprising administering to the subject an effective amount of a nanoparticle comprising a Beta coronavirus (e.g., MERS-CoV, SARS-CoV-1, or SARS-CoV-2) antigen (e.g. a RBD-NP) and an effective amount of an adjuvantation system comprising an OIW.

In some embodiments, the adjuvantation system (e.g., an OIW) is administered separately from the Beta coronavirus antigen (e.g., an antigen nanoparticle). In some embodiments, the adjuvantation system (e.g., an OIW) is administered prior to administering the Beta coronavirus antigen (e.g., an antigen nanoparticle). In some embodiments, the adjuvantation system (e.g., an OIW) is administered after administering the Beta coronavirus antigen (e.g., an antigen nanoparticle). In some embodiments, the adjuvantation system (e.g., an OIW) and the Beta coronavirus antigen (e.g., an antigen nanoparticle) are administered simultaneously. In some embodiments, the adjuvantation system (e.g., an OIW) and the Beta coronavirus antigen (e.g., an antigen nanoparticle) are administered as an admixture.

A “subject” to which administration is contemplated refers to a human (i.e., male or female of any age group, e.g., pediatric subject (e.g., infant, child, or adolescent) or adult subject (e.g., young adult, middle-aged adult, or senior (i.e., elderly) adult)) or non-human animal. In some embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In some embodiments, the non-human animal is a fish, reptile, or amphibian. The non-human animal may be a male or female at any stage of development. The non-human animal may be a transgenic animal or genetically engineered animal. A “subject in need thereof” refers to a subject (e.g., a human subject or a non-human mammal) in need of treatment of infection by a Beta coronavirus (e.g., a subject having MERS, SARS or COVID19) or in need of reducing the risk of developing an infection by Beta coronavirus (e.g., MERS-CoV, SARS-CoV-1, or SARS-CoV-2). In some embodiments, administering the Beta coronavirus (e.g., MERS-CoV, SARS-CoV-1, or SARS-CoV-2) antigen (e.g., an antigen nanoparticle) and the adjuvantation system (e.g., an OIW) described herein to a subject having Beta coronavirus (e.g., MERS-CoV, SARS-CoV-1, or SARS-CoV-2) infection treats (has a therapeutic use for) the disease (MERS, SARS or COVID-19). In some embodiments, administering the antigen (e.g., an antigen nanoparticle) and the adjuvantation system (e.g., an OIW) described herein to a subject at risk of developing an infection by a Beta coronavirus (e.g., MERS-CoV, SARS-CoV-1, or SARS-CoV-2) reduces the likelihood (e.g., by 20%, 30%, 40%, 50%, 60%, 70%, 80%, 90%, 95%, 99% or more) of the subject developing the infection (prophylactic use).

In some embodiments, the subject is a human subject, e.g., a human neonate, infant, child, adult, or elderly. In particular, the present disclosure demonstrates the immune enhancing effects of the antigen (e.g., antigen nanoparticle) and adjuvantation system described herein (e.g., an OIW) in adult (“young”) and elderly (“aged”) subjects.

In some embodiments, immunization for human subjects that are more than 28-days old (e.g., 1 month, 2 months, 3 months, 4 months, 5 months, 6 months, 7 months, 8 months, 9 months, 10 months, 11 months, 12 months, 2 years, 3 years, 4 years, 5 years, 10 years, 11 years, 12 years, 13 years, 14 years, 15 years, 16 years, 17 years old) is contemplated. In some embodiments, the human subject is an adult (e.g., more than 18 years old). In some embodiments, the human subject is an elderly (e.g., more than 60 years old). In some embodiments, the human subject is more than 65-years of age (e.g., 60 years, 65 years, 70 years, 75 years, 80 years, 85 years, 90 years, 95 years, 100 years, or more than 100 years old). In some embodiments, the human subject receives one or two doses of the vaccine described herein after 65-years of age.

In some embodiments, immunization of younger human subjects is contemplated. In some embodiments the subject is a human infant, or a human neonate (less than 28 days of age). In some embodiments, the human infant is less than 28 days of age at the time of administration (vaccination). In some embodiments, the human infant is less than 4 days of age at the time of administration (vaccination). In some embodiments, the human infant is less than 2 days of age at the time of administration (vaccination). In some embodiments, the human infant is less than 24 hours of age at the time of administration (vaccination). In some embodiments, the administration (vaccination) occurs at birth. In some embodiments, a human infant (less than 28 days of age) receives 1 or 2 doses of the vaccine described herein. In some embodiments, the human infant receives one dose before 28-days of age (e.g., 2, 3, 4, 5, 6, 7, 8, 9, 10, 11, 12, 13, 14, 15, 16, 17, 18, 19, 20 days of age) and a second dose before or at 28-days of age. In some embodiments, the human subject receives one dose at 2 months, 4 months, or 6 months of age, and a second dose after the first dose at 2 months, 4 months, or 6 months of age. In some embodiments, a human subject receives a second dose before or equal to 6-months of age (e.g., 1, 2, 3, 4, 5, 6 months of age). In some embodiments, the administration occurs when the human infant is 2 months, 4 months, and 6 months of age. In some embodiments, a human subject receives a second dose after 6-months of age (e.g., 1 year, 2 years, 3 years of age).

In some embodiments, a human subject receives 1, 2, or more than 2 doses of the vaccine described herein. In some embodiments, a human infant receives one dose before 28 days of age (e.g., 2, 3, 4, 5, 6, 7, 8, 9, 10, 11, 12, 13, 14, 15, 16, 17, 18, 19, 20 days of age) and a second dose before or after 28-days of age. In some embodiments, a human subject receives one dose before 60 years of age and a second dose before, at, or after 60 years of age (e.g., 60, 65, 70, 75, 80, 85, 90, 95, 100, or more than 100 years of age, or any age therebetween as if explicitly recited). In some embodiments, a human subject receives a second dose of the vaccine 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years or more after receiving the first dose.

In some embodiments, the human subject has an undeveloped (e.g., an infant or a neonate), weak (an elderly), or compromised immune system. Immunocompromised subjects include, without limitation, subjects with primary immunodeficiency or acquired immunodeficiency such as those suffering from sepsis, HIV infection, and cancers, including those undergoing chemotherapy and/or radiotherapy. In some embodiments, the human subject has an underlying condition that renders them more susceptible to Beta coronavirus (e.g., MERS-CoV, SARS-CoV-1, or SARS-CoV-2) infection. In some embodiments, the human subject is immunocompromised, immunosenescent, has a chronic disease such as, but not limited to, chronic lung disease, asthma, cardiovascular disease, cancer, obesity, diabetes, chronic kidney disease, and/or liver disease, is frail (e.g., has frailty syndrome), or is malnourished.

In some embodiments, the subject is a companion animal (a pet). The use of the immunogenic compositions (e.g., vaccine compositions) described herein in veterinary vaccine is also within the scope of the present disclosure. “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., ferrets, pigs, rats, mice, guinea pigs, and hamsters), rabbits, or non-human primates.

Once administered, the immunogenic composition (e.g., vaccine composition) described herein elicits an immune response in the subject. In some embodiments, the immune response is an innate immune response. In some embodiments, the immune response is an adaptive immune response specific to the antigen in the composition or vaccine. In some embodiments, the immunogenic composition (e.g., vaccine composition) described herein activates B cell immunity. In some embodiments, the immunogenic composition (e.g., vaccine composition) elicits production of antibodies against the antigen. In some embodiments, the immunogenic composition (e.g., vaccine composition) activates cytotoxic T cells specific to the antigen.

In some embodiments, the Beta coronavirus antigen (e.g., an antigen nanoparticle) and the adjuvantation system (e.g., an OIW) described herein, whether administered to a subject separately or as an admixture, enhance the innate immune response in the subject, compared to when the antigen is administered alone or in the absence of administration. In some embodiments, the Beta coronavirus antigen (e.g., an antigen nanoparticle) and the adjuvantation system (e.g., an OIW) activates innate immune cells (e.g., macrophages, dendritic cells, natural killer cells, neutrophils). In some embodiments, the number of innate immune cells that are activated is increased by at least 20% in the presence of the Beta coronavirus antigen (e.g., an antigen nanoparticle) and the adjuvantation system (e.g., an OIW) described herein, compared to when the antigen is administered alone or in the absence of administration. For example, the number of innate immune cells that are activated may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the Beta coronavirus antigen (e.g. an antigen nanoparticle) and the adjuvantation system (e.g., an OIW) described herein, compared to when the antigen is administered alone or in the absence of administration. In some embodiments, the number of innate immune cells that are activated is increased by 20%, 30%, 40%, 50%, 60%, 70%, 80%, 90%, 100%, 2-fold, 5-fold, 10-fold, 100-fold, 1000-fold or more, in the presence of the Beta coronavirus antigen (e.g. an antigen nanoparticle) and the adjuvantation system (e.g., an OIW) described herein, compared to when the antigen is administered alone or in the absence of administration.

In some embodiments, the Beta coronavirus antigen (e.g., an antigen nanoparticle) and the adjuvantation system (e.g., an OIW) described herein, whether administered to a subject separately or as an admixture, enhance the production of a proinflammatory cytokine (e.g., IL-2, IL-6, IL-10, TNF, IFNα, IFNγ, CCL3, CXCL8, GM-CSF) in the subject. In some embodiments, the level of proinflammatory cytokines (e.g., IL-2, IL-6, IL-10, TNF, IFNα, IFNγ, CCL3, CXCL8, GM-CSF) is increased by at least 20% in the presence of the Beta coronavirus antigen (e.g., an antigen nanoparticle) and the adjuvantation system (e.g., an OIW) described herein, compared to when the antigen is administered alone or in the absence of administration. For example, the level of proinflammatory cytokines (e.g., IL-2, IL-6, IL-10, TNF, IFNα, IFNγ, CCL3, CXCL8, GM-CSF) may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the Beta coronavirus antigen (e.g. an antigen nanoparticle) and the adjuvantation system (e.g., an OIW) described herein, compared to when the antigen is administered alone or in the absence of administration. In some embodiments, the level of proinflammatory cytokines (e.g., IL-2, IL-6, IL-10, TNF, IFNα, IFNγ, CCL3, CXCL8, GM-CSF) is increased by 20%, 30%, 40%, 50%, 60%, 70%, 80%, 90%, 100%, 2-fold, 5-fold, 10-fold, 100-fold, 1000-fold or more, in the presence of the Beta coronavirus antigen (e.g. an antigen nanoparticle) and the adjuvantation system (e.g., an OIW) described herein, compared to when the antigen is administered alone or in the absence of administration.

In some embodiments, the Beta coronavirus antigen (e.g., an antigen nanoparticle) and the adjuvantation system (e.g., an OIW) described herein, whether administered to a subject separately or as an admixture, elicit the expression of interferon (IFN)-stimulated genes (e.g., type I IFN-stimulated genes, such as CXCL19, IFIT2, and RSAD2) in the subject. In some embodiments, expression of IFN-stimulated genes is increased by at least 20% in the presence of the Beta coronavirus antigen (e.g., an antigen nanoparticle) and the adjuvantation system (e.g., an OIW) described herein, compared to when the antigen is administered alone or in the absence of administration. For example, expression of IFN-stimulated genes (e.g., type I IFN-stimulated genes, such as CXCL19, IFIT2, and RSAD2) may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the Beta coronavirus antigen (e.g., an antigen nanoparticle) and the adjuvantation system (e.g., an OIW) described herein, compared to when the antigen is administered alone or in the absence of administration. In some embodiments, expression of IFN-stimulated genes (e.g., type I IFN-stimulated genes, such as CXCL19, IFIT2, and RSAD2) is increased by 20%, 30%, 40%, 50%, 60%, 70%, 80%, 90%, 100%, 2-fold, 5-fold, 10-fold, 100-fold, 1000-fold or more, in the presence of the Beta coronavirus antigen (e.g. an antigen nanoparticle) and the adjuvantation system (e.g., an OIW) described herein, compared to when the antigen is administered alone or in the absence of administration.

In some embodiments, the Beta coronavirus antigen (e.g., an antigen nanoparticle) and the adjuvantation system (e.g., an OIW) described herein, whether administered to a subject separately or as an admixture, enhance innate immune memory (also referred to as trained immunity). “Innate immune memory” confers heterologous immunity that provides broad protection against a range of pathogens. In some embodiments, the innate immune memory is increased by at least 20% in the presence of the Beta coronavirus antigen (e.g., an antigen nanoparticle) and the adjuvantation system (e.g., an OIW) described herein, compared to when the antigen is administered alone or in the absence of administration. For example, the innate immune memory may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the Beta coronavirus antigen (e.g. an antigen nanoparticle) and the adjuvantation system (e.g., an OIW) described herein, compared to when the antigen is administered alone or in the absence of administration. In some embodiments, the innate immune memory is increased by 20%, 30%, 40%, 50%, 60%, 70%, 80%, 90%, 100%, 2-fold, 5-fold, 10-fold, 100-fold, 1000-fold or more, in the presence of the Beta coronavirus antigen (e.g., an antigen nanoparticle) and the adjuvantation system (e.g., an OIW) described herein, compared to when the antigen is administered alone or in the absence of administration.

In some embodiments, the Beta coronavirus antigen (e.g., an antigen nanoparticle) and the adjuvantation system (e.g., an OIW) described herein, whether administered to a subject separately or as an admixture, enhance the antigen-specific immune response against the Beta coronavirus (e.g., MERS-CoV, SARS-CoV-1, or SARS-CoV-2) antigen or against the Beta coronavirus (e.g., MERS-CoV, SARS-CoV-1, or SARS-CoV-2), compared to when the antigen is administered alone or in the absence of administration. In some embodiments, the Beta coronavirus antigen (e.g., an antigen nanoparticle) and the adjuvantation system (e.g., an OIW) enhances the production of antigen-specific antibody titer (e.g., by at least 20%) in the subject, compared to when the antigen is administered alone or in the absence of administration. For example, the Beta coronavirus antigen (e.g. an antigen nanoparticle) and the adjuvantation system (e.g., an OIW) may enhance the production of antigen-specific antibody titer by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more in the subject, compared to when the antigen is administered alone or in the absence of administration. In some embodiments, the Beta coronavirus antigen (e.g. an antigen nanoparticle) and the adjuvantation system (e.g., an OIW) enhances the production of antigen-specific antibody titer by 20%, 30%, 40%, 50%, 60%, 70%, 80%, 90%, 100%, 2-fold, 5-fold, 10-fold, 100-fold, 1000-fold or more, in the presence of the Beta coronavirus antigen (e.g. an antigen nanoparticle) and the adjuvantation system (e.g., an OIW), compared to when the antigen is administered alone or in the absence of administration. One skilled in the art is familiar with how to evaluate the level of an antibody titer, e.g., by ELISA. In some embodiments, the antigen-specific antibody for which production is enhanced is an immunoglobulin A (IgA), immunoglobulin D (IgG), immunoglobulin E (IgE), immunoglobulin G (IgG), or immunoglobulin M (IgM). In some embodiments, the antigen-specific antibody is an IgG. In some embodiments, the antigen-specific antibody is a subclass 1 IgG (IgG1), subclass 2 IgG (IgG2), subclass 3 IgG (IgG3), or subclass 4 IgG (IgG4).

In some embodiments, the Beta coronavirus antigen (e.g., an antigen nanoparticle) and the adjuvantation system (e.g., an OIW) described herein, whether administered to a subject separately or as an admixture, enhances the production of antigen-specific antibodies that neutralize (i.e., render non-infectious) Beta coronavirus particles. In some embodiments, the Beta coronavirus antigen (e.g. an antigen nanoparticle) and the adjuvantation system (e.g., an OIW) enhance the neutralizing antibody titer by 20%, 30%, 40%, 50%, 60%, 70%, 80%, 90%, 100%, 2-fold, 5-fold, 10-fold, 100-fold, 1000-fold or more, in the presence of the Beta coronavirus antigen (e.g. an antigen nanoparticle) and the adjuvantation system (e.g., an OIW), compared to when the antigen is administered alone or in the absence of administration. In some embodiments, the Beta coronavirus antigen (e.g., an antigen nanoparticle) and the adjuvantation system (e.g., an OIW) enhance the antigen-specific antibody titer capable of neutralizing a particular Beta coronavirus variant, compared to when the antigen is administered alone or in the absence of administration. In some embodiments, the variant is wild-type MERS-CoV, SARS-CoV-1, or SARS-CoV-2. In some embodiments, the variant is a Beta coronavirus variant that is not a wild-type variant. In some embodiments, the variant is a variant of SARS-CoV-2 that is not wild-type SARS-CoV-2, such as, but not limited to, B.1.1.7, B.1.351, P.1, B.1.427, B.1.429, B.1.526, B.1.526.1, B.1.525, P.2, B.1.617, B.1.617.1, B.1.617.2, or B.1.617.3 SARS-CoV-2.

In some embodiments, the Beta coronavirus antigen (e.g., an antigen nanoparticle) and the adjuvantation system (e.g., an OIW) described herein, whether administered to a subject separately or as an admixture, polarize the innate and adaptive immune response by shaping the pattern of cytokine and/or chemokine responses toward T helper 1 (Th1) immunity, important for host defense against intracellular pathogens. In some embodiments, the Beta coronavirus antigen (e.g., an antigen nanoparticle) and the adjuvantation system (e.g., an OIW) described herein, whether administered to a subject separately or as an admixture, polarize the innate immune response toward T follicular helper (Tfh) cell immunity.

In some embodiments, the Beta coronavirus antigen (e.g., an antigen nanoparticle) and the adjuvantation system (e.g., an OIW) described herein, whether administered to a subject separately or as an admixture, enhance the inhibition of interaction between angiotensin-converting enzyme 2 (ACE2) expressed by a subject and Beta coronavirus spike protein, compared to when the antigen is administered alone or in the absence of administration. For example, the Beta coronavirus antigen (e.g. an antigen nanoparticle) and the adjuvantation system (e.g., an OIW) may enhance the inhibition of interaction between ACE2 expressed by a subject and Beta coronavirus spike protein by 20%, 30%, 40%, 50%, 60%, 70%, 80%, 90%, 100%, 2-fold, 5-fold, 10-fold, 100-fold, 1000-fold or more in the presence of the Beta coronavirus antigen (e.g. an antigen nanoparticle) and the adjuvantation system (e.g., an OIW), compared to when the antigen is administered alone or in the absence of administration. In the presence of Beta coronavirus antigen (e.g., an antigen nanoparticle) and the adjuvantation system (e.g., an OIW), interaction between ACE2 expressed by a subject and Beta coronavirus spike protein may be reduced by 10%, 20%, 30%, 40%, 50%, 60%, 70%, 80%, 90%, 95%, 99%, or more than 99%, compared to when the antigen is administered alone or in the absence of administration.

In some embodiments, the Beta coronavirus antigen (e.g., an antigen nanoparticle) and the adjuvantation system (e.g., an OIW) described herein, whether administered to a subject separately or as an admixture, prolong the effect of a vaccine (e.g., by at least 20%) in the subject, compared to when the antigen is administered alone or in the absence of administration. For example, the Beta coronavirus antigen (e.g. an antigen nanoparticle) and the adjuvantation system (e.g., an OIW) may prolong the effect of a vaccine by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more in the subject, compared to when the antigen is administered alone or in the absence of administration. In some embodiments, the Beta coronavirus antigen (e.g. an antigen nanoparticle) and the adjuvantation system (e.g., an OIW) prolongs the effect of a vaccine by 20%, 30%, 40%, 50%, 60%, 70%, 80%, 90%, 100%, 2-fold, 5-fold, 10-fold, 100-fold, 1000-fold or more, in the presence of the Beta coronavirus antigen (e.g. an antigen nanoparticle) and the adjuvantation system (e.g., an OIW), compared to when the antigen is administered alone or in the absence of administration.

In some embodiments, the Beta coronavirus antigen (e.g., an antigen nanoparticle) and the adjuvantation system (e.g., an OIW) described herein, whether administered to a subject separately or as an admixture, increase the rate of (accelerates) an immune response, compared to when the antigen is administered alone or in the absence of administration. For example, the Beta coronavirus antigen (e.g. an antigen nanoparticle) and the adjuvantation system (e.g., an OIW) may increase the rate of an immune response by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more in the subject, compared to when the antigen is administered alone or in the absence of administration. In some embodiments, the Beta coronavirus antigen (e.g. an antigen nanoparticle) and the adjuvantation system (e.g., an OIW) increases the rate of an immune response by 20%, 30%, 40%, 50%, 60%, 70%, 80%, 90%, 100%, 2-fold, 5-fold, 10-fold, 100-fold, 1000-fold or more in the presence of the Beta coronavirus antigen (e.g. an antigen nanoparticle) and the adjuvantation system (e.g., an OIW), compared to when the antigen is administered alone or in the absence of administration. The expression “increase the rate of immune response” means that it takes less time for the immune system of a subject to react to the presence of an invading Beta coronavirus (e.g., MERS-CoV, SARS-CoV-1, or SARS-CoV-2).

In some embodiments, the antigen (e.g., an antigen nanoparticle) produces a same level of immune response against the Beta coronavirus (e.g., MERS-CoV, SARS-CoV-1, or SARS-CoV-2) antigen at a lower dose in the presence of the adjuvantation system (e.g., an OIW), compared to without the adjuvantation system or when the Beta coronavirus antigen is administered alone. In some embodiments, the amount of Beta coronavirus antigen (e.g., an antigen nanoparticle) needed to produce the same level of immune response is reduced by at least 20% in the presence of the adjuvantation system (e.g., an OIW), compared to without the adjuvantation system or when the Beta coronavirus antigen is administered alone. For example, the amount of antigen needed to produce the same level of immune response may be reduced by at least 20%, at least 30%, at least 40%, at least 50%, at least 60%, at least 70%, at least 80%, at least 90%, at least 95%, at least 99% or more in the presence of the adjuvantation system, compared to without the adjuvantation system or when the Beta coronavirus antigen is administered alone. In some embodiments, the amount of antigen needed to produce the same level of immune response is reduced by 20%, 30%, 40%, 50%, 60%, 70%, 80%, 90%, 95%, 99% or more, in the presence of the adjuvantation system, compared to without the adjuvantation system or when the Beta coronavirus antigen is administered alone.

In some embodiments where the antigen is an antigen nanoparticle, the antigen produces a same level of immune response against the Beta coronavirus (e.g., MERS-CoV, SARS-CoV-1, or SARS-CoV-2) antigen at a lower dose compared to when the Beta coronavirus antigen is administered without association with or in the absence of the nanoparticle. In some embodiments, the amount of antigen nanoparticle needed to produce the same level of immune response is reduced by at least 20% compared to when the Beta coronavirus antigen is administered without association with or in the absence of the nanoparticle. For example, the amount of antigen nanoparticle needed to produce the same level of immune response may be reduced by at least 20%, at least 30%, at least 40%, at least 50%, at least 60%, at least 70%, at least 80%, at least 90%, at least 95%, at least 99% or more compared to when the Beta coronavirus antigen is administered without association with or in the absence of the nanoparticle. In some embodiments, the amount of antigen nanoparticle needed to produce the same level of immune response is reduced by 20%, 30%, 40%, 50%, 60%, 70%, 80%, 90%, 95%, 99% or more compared to when the Beta coronavirus antigen is administered without association with or in the absence of the nanoparticle.

In some embodiments where the antigen is an antigen nanoparticle, the antigen enhances production of antigen-specific antibodies against the Beta coronavirus (e.g., MERS-CoV, SARS-CoV-1, or SARS-CoV-2) antigen at a lower dose compared to when the Beta coronavirus antigen is administered without association with or in the absence of the nanoparticle. In some embodiments, the amount of antigen nanoparticle needed to enhance production of the same level of antigen-specific antibodies is reduced by at least 20% compared to when the Beta coronavirus antigen is administered without association with or in the absence of the nanoparticle. For example, the amount of antigen nanoparticle needed to enhance production of the same level of antigen-specific antibodies may be reduced by at least 20%, at least 30%, at least 40%, at least 50%, at least 60%, at least 70%, at least 80%, at least 90%, at least 95%, at least 99% or more compared to when the Beta coronavirus antigen is administered without association with or in the absence of the nanoparticle. In some embodiments, the amount of antigen nanoparticle needed to enhance production of the same level of antigen-specific antibodies is reduced by 20%, 30%, 40%, 50%, 60%, 70%, 80%, 90%, 95%, 99% or more compared to when the Beta coronavirus antigen is administered without association with or in the absence of the nanoparticle.

In some embodiments where the antigen is an antigen nanoparticle, the antigen prolongs the protective effect against the Beta coronavirus (e.g., MERS-CoV, SARS-CoV-1, or SARS-CoV-2) in a subject compared to when the Beta coronavirus antigen is administered without association with or in the absence of the nanoparticle. In some embodiments, the antigen nanoparticle prolongs the protective effect against the Beta coronavirus (e.g., MERS-CoV, SARS-CoV-1, or SARS-CoV-2) by at least 10% compared to when the Beta coronavirus antigen is administered without association with or in the absence of the nanoparticle. For example, the antigen nanoparticle may prolong the protective effect against the Beta coronavirus (e.g., MERS-CoV, SARS-CoV-1, or SARS-CoV-2) by at least 10%, by at least 20%, by at least 30%, by at least 40%, by at least 50%, by at least 60%, by at least 70%, by at least 80%, by at least 90%, by at least 100%, by at least 2-fold, by at least 5-fold, by at least 10-fold, by at least 100-fold, by at least 1000-fold or more, compared to an equal amount of the Beta coronavirus antigen without association with or in the absence of the nanoparticle. In some embodiments, the antigen nanoparticle prolongs the protective effect against the Beta coronavirus (e.g., MERS-CoV, SARS-CoV-1, or SARS-CoV-2) by 10%, 20%, 30%, 40%, 50%, 60%, 70%, 80%, 90%, 100%, 2-fold, 5-fold, 10-fold, 100-fold, 1000-fold or more compared to an equal amount of the Beta coronavirus antigen without association with or in the absence of the nanoparticle.

The prophylactic or therapeutic use of the Beta coronavirus antigen (e.g., an antigen nanoparticle) and the adjuvantation system (e.g., an OIW), or the immunogenic composition (e.g., vaccine composition) described herein is also within the scope of the present disclosure. In some embodiments, the composition or immunogenic composition (e.g., vaccine composition) described herein are used in methods of vaccinating a subject by prophylactically administering to the subject an effective amount of the composition or immunogenic composition (e.g., vaccine composition) described herein. “Vaccinating a subject” refer to a process of administering an immunogen, typically an antigen formulated into a vaccine, to the subject in an amount effective to increase or activate an immune response against the Beta coronavirus antigen (e.g., MERS-COV, SARS-COV-1, SARS-COV-2) and, thus, against Beta coronavirus (e.g., MERS-COV, SARS-COV-1, SARS-COV-2). In some embodiments, the terms do not require the creation of complete immunity against SARS-CoV. In some embodiments, the terms encompass a clinically favorable enhancement of an immune response toward the Beta coronavirus antigen or pathogen. Methods for immunization, including formulation of an immunogenic composition (e.g., vaccine composition) and selection of doses, routes of administration and the schedule of administration (e.g., primary dose and one or more booster doses), are well known in the art. In some embodiments, vaccinating a subject reduces the risk of developing Beta coronavirus (e.g., MERS-CoV, SARS-CoV-1, or SARS-CoV-2) infection and the resulting disease (e.g., MERS, SARS and/or COVID19)

In some embodiments, the immunogenic compositions (e.g., vaccine composition) described herein are formulated for administration to a subject. In some embodiments, the composition or immunogenic composition (e.g., vaccine composition) further comprises a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the composition or immunogenic composition (e.g., vaccine composition) described herein also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The immunogenic composition (e.g., vaccine composition) described herein may conveniently be presented in unit dosage form and may be prepared by any of the methods well-known in the art of pharmacy. The term “unit dose” when used in reference to a composition or immunogenic composition (e.g., vaccine composition) described herein of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the composition or immunogenic compositions (e.g., vaccine composition) described herein may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the composition or immunogenic composition (e.g., vaccine composition) described herein can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, an elixir, or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide-based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the immunogenic composition (e.g., vaccine composition) described herein used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The cyclic Psap peptide and/or the composition or immunogenic composition (e.g., vaccine composition) described herein ordinarily will be stored in lyophilized form or as an aqueous solution if it is highly stable to thermal and oxidative denaturation. The pH of the preparations typically will be about from 6 to 8, although higher or lower pH values can also be appropriate in certain instances. The chimeric constructs of the present disclosure can be used as vaccines by conjugating to soluble immunogenic carrier molecules. Suitable carrier molecules include protein, including keyhole limpet hemocyanin, which is a preferred carrier protein. The chimeric construct can be conjugated to the carrier molecule using standard methods. (Hancock et al., “Synthesis of Peptides for Use as Immunogens,” in Methods in Molecular Biology: Immunochemical Protocols, Manson (ed.), pages 23-32 (Humana Press 1992)).

In some embodiments, the present disclosure contemplates an immunogenic composition (e.g., vaccine composition) comprising a pharmaceutically acceptable injectable vehicle. The vaccines of the present disclosure may be administered in conventional vehicles with or without other standard carriers, in the form of injectable solutions or suspensions. The added carriers might be selected from agents that elevate total immune response in the course of the immunization procedure.

Liposomes have been suggested as suitable carriers. The insoluble salts of aluminum, that is aluminum phosphate or aluminum hydroxide, have been utilized as carriers in routine clinical applications in humans. Polynucleotides and polyelectrolytes and water-soluble carriers such as muramyl dipeptides have been used.

Preparation of injectable vaccines of the present disclosure, includes mixing the immunogenic composition (e.g., vaccine composition) with muramyl dipeptides or other carriers. The resultant mixture may be emulsified in a mannide monooleate/squalene or squalane vehicle. Four parts by volume of squalene and/or squalane are used per part by volume of mannide monooleate. Methods of formulating immunogenic composition (e.g., vaccine composition)s are well-known to those of ordinary skill in the art. (Rola, Immunizing Agents and Diagnostic Skin Antigens. In: Remington's Pharmaceutical Sciences, 18th Edition, Gennaro (ed.), (Mack Publishing Company 1990) pages 1389-1404).

Additional pharmaceutical carriers may be employed to control the duration of action of a vaccine in a therapeutic application. Control release preparations can be prepared through the use of polymers to complex or adsorb chimeric construct. For example, biocompatible polymers include matrices of poly(ethylene-co-vinyl acetate) and matrices of a polyanhydride copolymer of a stearic acid dimer and sebacic acid. (Sherwood et al. (1992) Bio/Technology 10: 1446). The rate of release of the chimeric construct from such a matrix depends upon the molecular weight of the construct, the amount of the construct within the matrix, and the size of dispersed particles. (Saltzman et al. (1989) Biophys. J. 55: 163; Sherwood et al, supra.; Ansel et al. Pharmaceutical Dosage Forms and Drug Delivery Systems, 5th Edition (Lea & Febiger 1990); and Gennaro (ed.), Remington's Pharmaceutical Sciences, 18th Edition (Mack Publishing Company 1990)). The chimeric construct can also be conjugated to polyethylene glycol (PEG) to improve stability and extend bioavailability times (e.g., Katre et al.; U.S. Pat. No. 4,766,106).

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. Prophylactic treatment refers to the treatment of a subject who is not and was not with a disease but is at risk of developing the disease or who was with a disease, is not with the disease, but is at risk of regression of the disease. In some embodiments, the subject is at a higher risk of developing the disease or at a higher risk of regression of the disease than an average healthy member of a population.

An “effective amount” of a composition described herein refers to an amount sufficient to elicit the desired biological response. An effective amount of a composition described herein may vary depending on such factors as the desired biological endpoint, the pharmacokinetics of the compound, the condition being treated, the mode of administration, and the age and health of the subject. In some embodiments, an effective amount is a therapeutically effective amount. In some embodiments, an effective amount is a prophylactic treatment. In some embodiments, an effective amount is the amount of a compound described herein in a single dose. In some embodiments, an effective amount is the combined amounts of a compound described herein in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a compound described herein, or a composition thereof, in or on a subject. The composition of the immunogenic composition (e.g., vaccine composition) described herein may be administered systemically (e.g., via intravenous injection) or locally (e.g., via local injection). In some embodiments, the composition of the immunogenic composition (e.g., vaccine composition) described herein is administered orally, intravenously, topically, intranasally, or sublingually. Parenteral administration is also contemplated. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, the composition is administered prophylactically.

In some embodiments, the composition or immunogenic composition (e.g., vaccine composition) is administered once or multiple times (e.g., 2, 3, 4, 5, or more times). For multiple administrations, the administrations may be done over a period of time (e.g., 6 months, a year, 2 years, 5 years, 10 years, or longer). In some embodiments, the composition or immunogenic composition (e.g., vaccine composition) is administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later).

### EXAMPLES

### Example 1—In Vitro Characterization of RBD-NP Reveals High Density Display Multimeric RBD

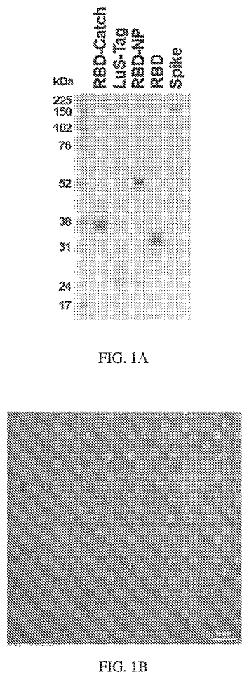

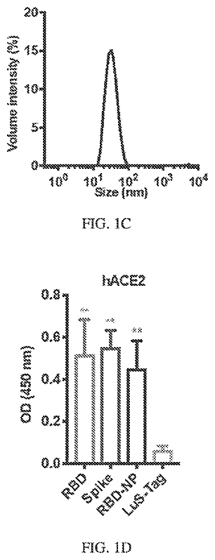

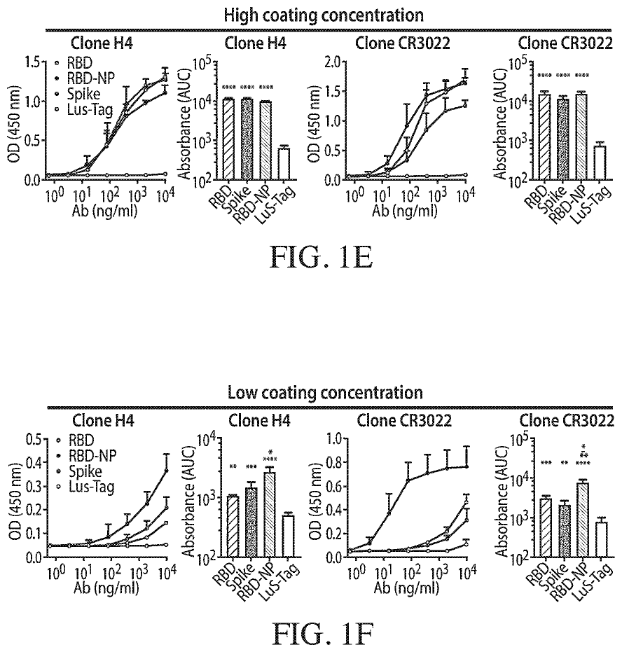

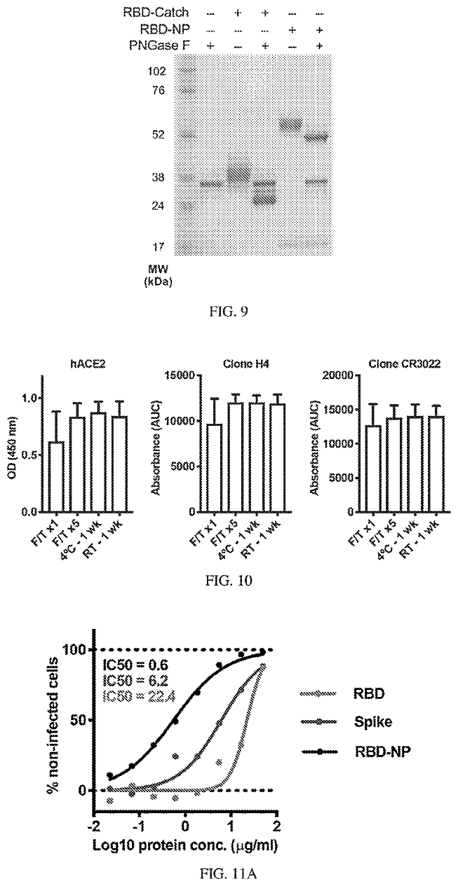

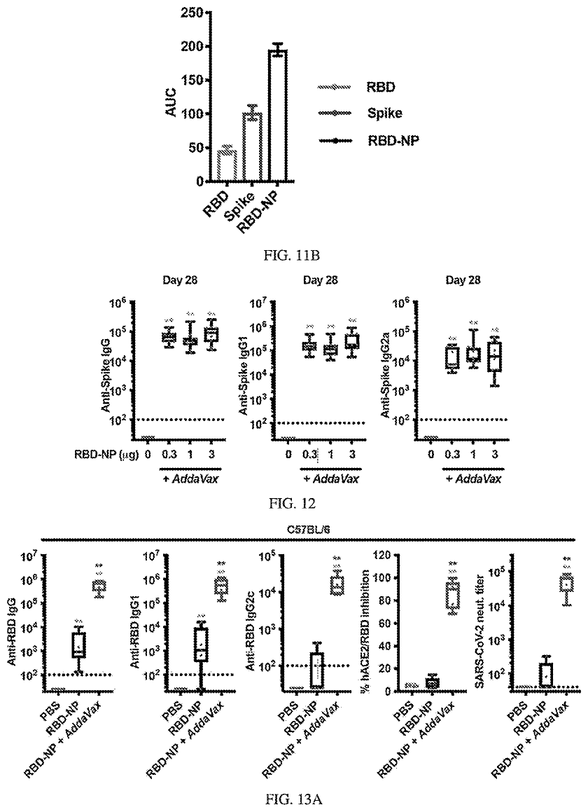

High density display of antigens onto protein NPs increases their immunogenicity and has been employed in several vaccine candidates against viral infections to elicit robust serum antigen-specific antibody titers (Singh, 2021). In order to assemble SARS-CoV-2 RBD onto a protein NP scaffold, the SpyTag/SpyCatcher conjugation system was used in which proteins fused with SpyTag and Spy Catcher spontaneously form stable isopeptide bonds (Brune et al., 2016). Briefly, the self-aggregating lumazine synthase (LuS) from the hyperthermophile “Aquifex aeolicus” was used for a protein NP scaffold (Zhang et al., 2001). RBD and LuS were modified with SpyCatcher (RBD-Catch) and SpyTag (LuS-Tag), respectively. SDS-PAGE analysis of RBD-Catch, LuS-Tag, RBD-NP (generated by conjugating RBD-Catch and LuS-Tag) under reducing conditions, as well as native RBD and Spike proteins, confirmed expected molecular weights of each protein (FIG. 1A). SDS-PAGE analysis demonstrated multiple bands of RDP-NP (FIG. 1A), which was likely due to heterogenous glycosylation patterns since doublets were no longer observed following Peptide-N-Glycosidase F (PNGase F) treatment (FIG. 9). Transmission electron microscopy analysis of RBD-NP revealed a ruffled border, suggesting efficient conjugation and display of RBD onto the protein scaffold, and homogeneous size (FIG. 1B). This conclusion was further confirmed by dynamic light scattering analysis, which indicated an average particle size of ˜30 nm (FIG. 1C). To confirm proper display of RBD onto NPs, ELISA plates were coated with RBD, Spike, RBD-NP, and LuS-Tag, and were assessed for binding to recombinant human ACE2 (hACE2) and two anti-RBD monoclonal antibodies (mAbs: clones H4 and CR3022). RBD-NP binding to hACE2, clones H4 and CR3022 was comparable to RBD and Spike while no binding to LuS-Tag was observed (FIGS. 1D and 1E). RBD-NP binding profiles remained unaltered under multiple storage conditions, namely 5 freeze/thaw cycles or storage for 1 week at 4° C. or room temperature (FIG. 10). Interestingly, by assessing binding to mAb clones H4 and CR3022 under lower coating concentrations of RBD, Spike, and RBD-NP preferential binding was observed to RBD-NP (FIG. 1F), suggesting that high density display of RBD onto NP increases antibody avidity. To explore another functional correlate of RBD-NP structure, a competition assay was performed in which Vero cells were incubated with SARS-CoV-2 in the absence or presence of multiple concentrations of RBD, Spike or RBD-NP. RBD-NP significantly reduced SARS-CoV-2 infection as assessed by IC₅₀ and AUC (FIGS. 11A and 11B), further supporting high density display of RBD onto NP.

### Example 2—Immunization with RBD-NP Elicits High Serum Anti-RBD Antibody Titers and SARS-CoV-2 Neutralizing Titers

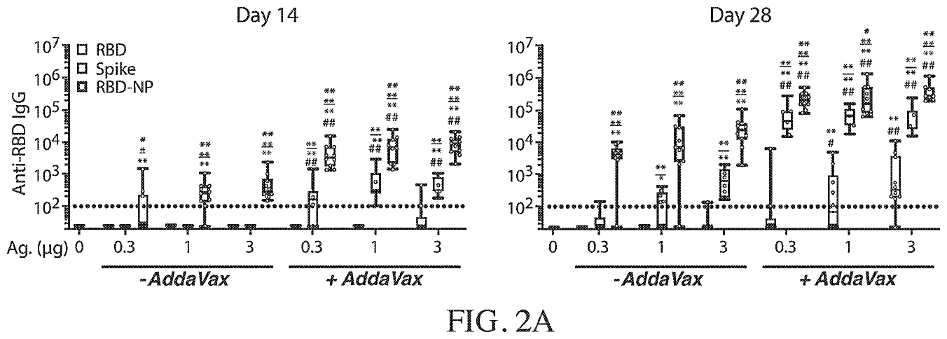

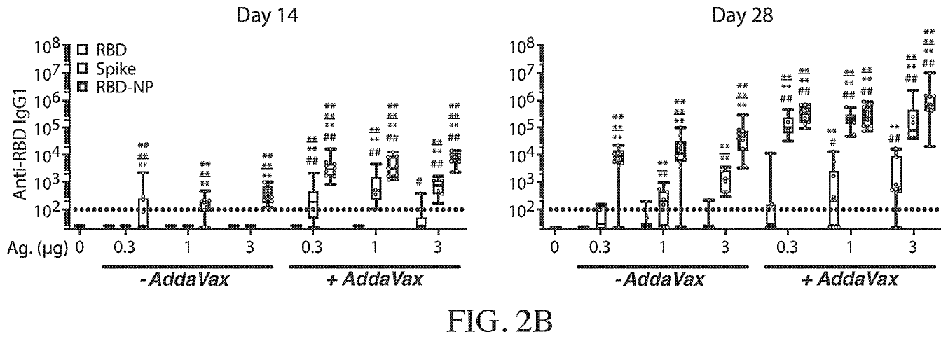

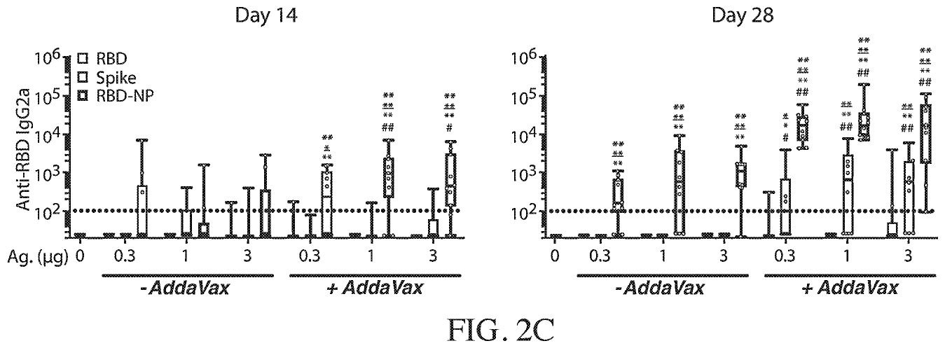

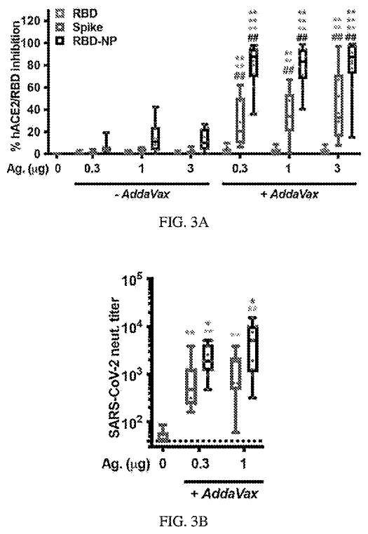

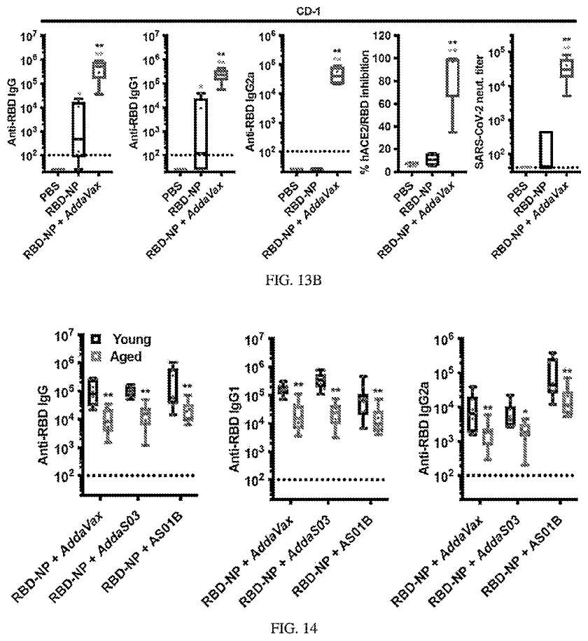

To assess whether RBD-NP increases RBD immunogenicity, BALB/c mice were immunized with multiple doses of RBD, Spike, or RBD-NP, alone or formulated with AddaVax using a prime (Day 0)-boost (Day 14) immunization schedule (FIGS. 2A-2C). As expected, formulation with AddaVax enhanced anti-RBD antibody titers compared to immunizations with non-formulated antigens. RBD-NP induced the greatest titers of anti-RBD IgG, IgG1 and IgG2a of all experimental antigens tested, especially at the lowest tested dose (0.3 μg) and even in the absence of AddaVax, thus showing a robust dose-sparing effect. Of note, anti-RBD antibodies elicited by immunization with RBD-NP also recognized native RBD on Spike (FIG. 12), which is key for SARS-CoV-2 neutralization. To confirm this point, a surrogate of virus neutralization test (sVNT) was performed to measure the degree of inhibition of RBD binding to hACE2 by immune sera, as well as a neutralization assay with live SARS-CoV-2 virus. In each assay, immunization with RBD-NP formulated with AddaVax induced higher levels of neutralization compared to immunization with Spike (FIGS. 3A and 3B), while immunization with monomeric RBD failed to elicit significant levels of neutralization in the sVNT (FIG. 3A). Of note, immunization with RBD-NP formulated with AddaVax elicited high levels of anti-RBD neutralizing antibodies in one additional inbred (C57BL/6) and one outbred (CD-1) mouse strain (FIGS. 13A-13B). Overall, these results show that multimeric RBD displayed on a NP significantly enhances its immunogenicity across multiple mouse strains, eliciting high levels of anti-RBD neutralizing antibodies with significant dose-sparing effect.

### Example 3—OIW Adjuvants Significantly Enhance RBD-NP Immunogenicity in Young and Aged Mice

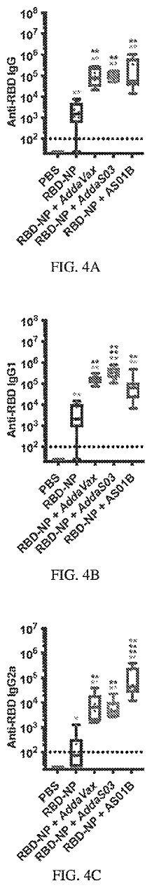

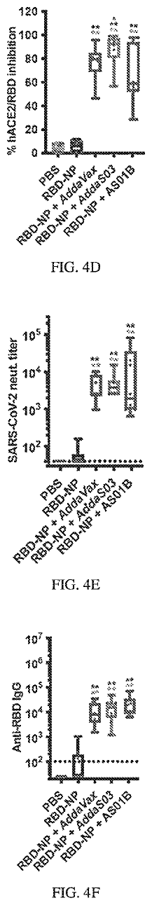

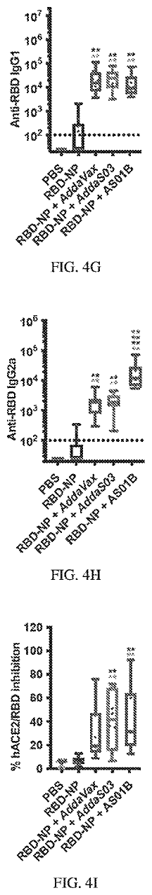

Adjuvants play a key role in enhancing antigen immunogenicity (Irvine et al., 2020; Nanishi et al., 2020; O'Hagan et al., 2020; Pulendran et al., 2021). The immunogenicity of RBD-NP was evaluated when formulated with different OIW emulsions, namely AddaVax and a AS03-like adjuvant, AddaS03 (Blom and Hilgers, 2004; Hilgers et al., 2017). AS01B (a liposome-based adjuvant containing monophosphoryl lipid A and saponin QS-21) was also tested as a clinical-grade benchmark adjuvant with potent immunostimulatory activity (Cunningham et al., 2016; Lal et al., 2015). RBD-NP formulations comprising these OIW adjuvants were tested in both young (3 months old) and aged (14 months old) mice to assess whether an optimized vaccine formulation could overcome impaired vaccine immunogenicity associated with immunosenescence in aged populations (Gustafson et al., 2020). Each of the tested adjuvanted RBD-NP vaccine formulations induced robust titers of anti-RBD neutralizing antibodies in young mice (FIGS. 4A-4E). Immunization of aged mice generally resulted in overall lower anti-RBD antibody titers compared to young mice (FIG. 14). Nevertheless, AddaVax, AddaS03, and AS01B substantially enhanced RBD-NP immunogenicity and induced anti-RBD antibody titers (FIGS. 4F-4H), inhibition of RBD binding to hACE2 (FIG. 4I) and SARS-CoV-2 neutralization (FIG. 4J) in aged mice.

### Example 4—Immunization with RBD-NP Formulated with OIW Adjuvants Elicits Cross-Neutralizing Antibodies

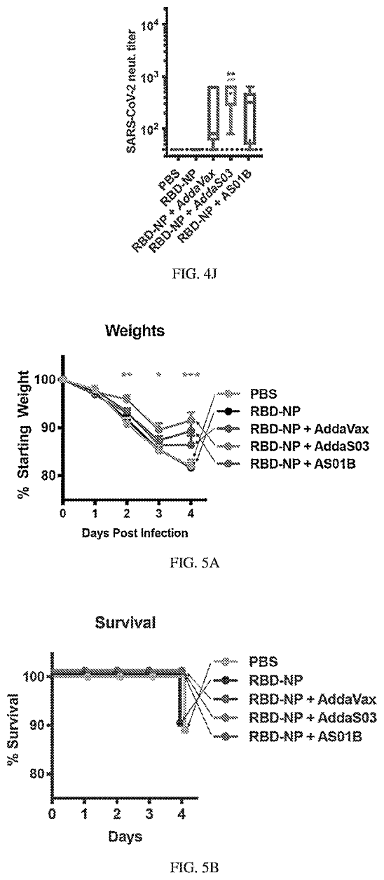

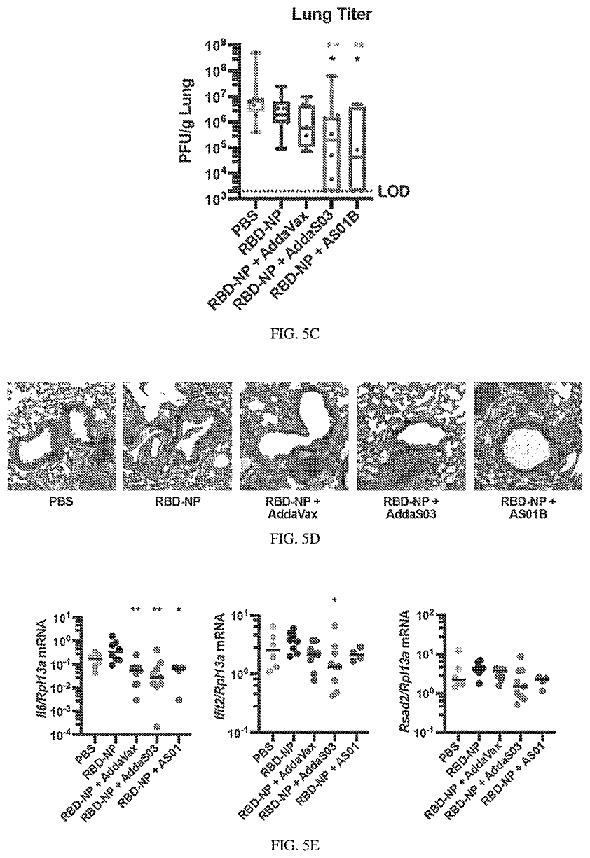

Neutralizing antibody titers are an important correlate of protection against SARS-CoV-2 infection (Corbett et al., 2021; McMahan et al., 2021). Therefore, it was assessed whether the high titers of neutralizing antibodies in mice immunized with OIW-adjuvanted RBD-NP translate into enhanced protection against SARS-CoV-2 over other adjuvants. To this end, aged BALBc mice were immunized with RBD-NP formulated with OIW, AddaVax, AddaS03, or AS01B and challenged with 10³ PFU of the mouse-adapted strain SARS-CoV-2 MA10 (Leist et al., 2020) (FIGS. 5A-5E). Mice immunized with RBD formulated with OIW were also included to assess relative protection induced by RBD-NP as compared to monomeric RBD. Strikingly, aged mice immunized with RBD-NP formulated OIW were protected from weight loss, whereas mice receiving non-adjuvanted RBD-NP or OIW-adjuvanted monomeric RBD showed significant weight loss comparable to the naive mice (FIG. 5A). Mice immunized with RBD-NP formulated with a benchmarking adjuvant, namely AddaVax, AddaS03, or AS01B, were partially protected. Survival rate (FIG. 5B), lung viral titers (FIG. 5C), lung histopathological analysis (FIG. 5D), and Il6, Ifit2, and Rsad2 gene expression in the lungs (FIG. 5E) further confirmed the reduced SARS-CoV-2 infection in aged mice immunized with RBD-NP formulated with OIW adjuvant.

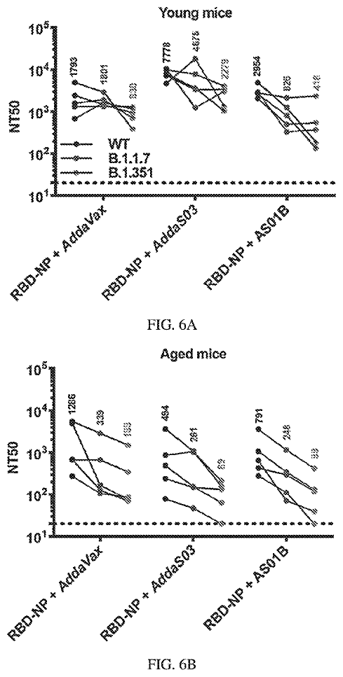

Since the beginning of the SARS-CoV-2 pandemic several SARS-CoV-2 variants such as B.1.1.7 and B.1.351 have emerged, the latter showing reduced neutralization by serum samples of convalescent or vaccinated subjects (Garcia-Beltran et al., 2021; Kuzmina et al., 2021; Shen et al., 2021). Neutralization of SARS-CoV-2 wild type (WT), B.1.17 and B.1.351 pseudoviruses was assessed using serum samples collected from young and aged mice immunized with RBD-NP formulated with AddaVax, AddaS03, or AS01B (FIGS. 6A-6B). Mice immunized with adjuvanted RBD-NP demonstrated neutralizing serum activity against B1.1.7 and B.1.351 variants, though with lower titer compared to SARS-CoV-2 WT.

### Example 5—OIW and AS01B RBD-NP Enhance CD4+ T Cell Responses in Young and Aged Mice

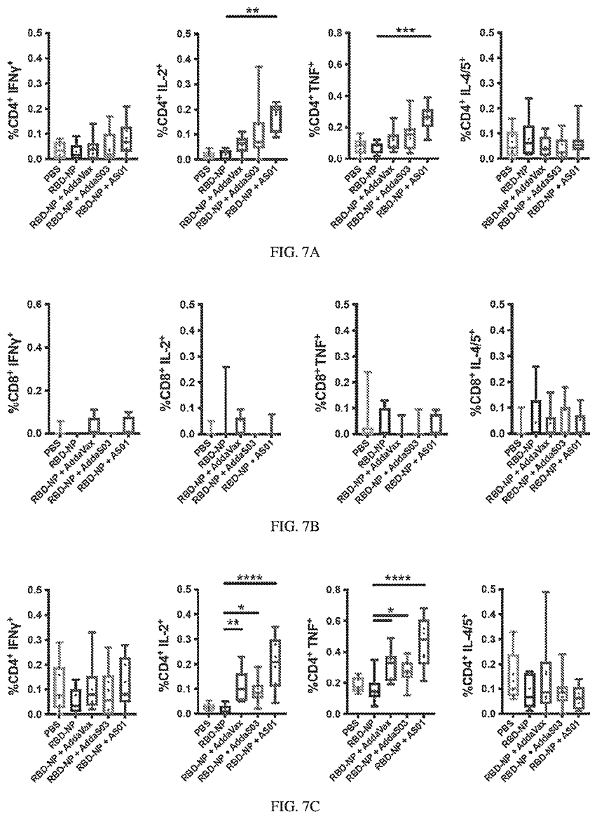

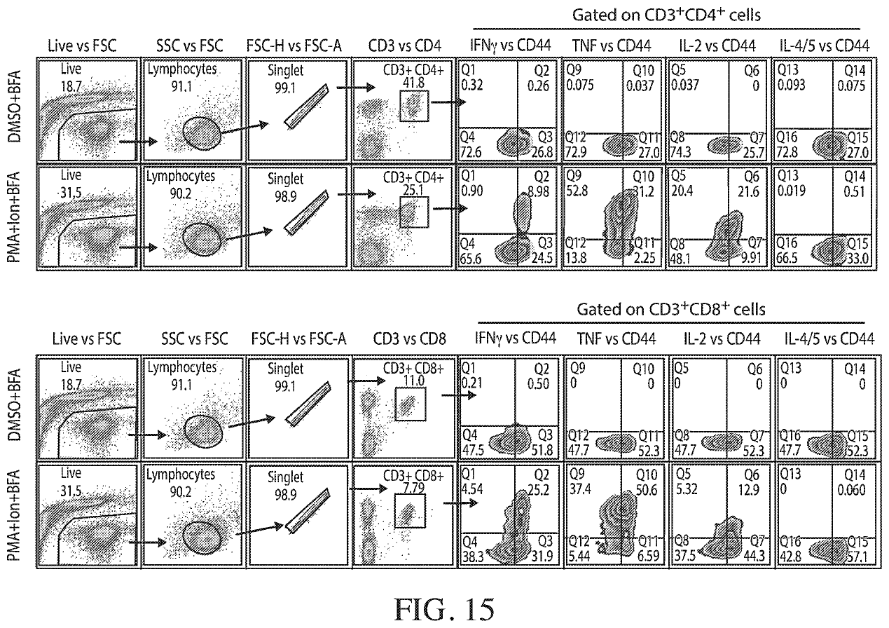

T cell responses induced by SARS-CoV-2 vaccines suppress viral replication and modulate disease severity (Israelow et al., 2021; McMahan et al., 2021; Tan et al., 2021a). Specific T cell responses were therefore analyzed by stimulating splenocytes derived from immunized young and aged mice with a SARS-CoV-2 spike RBD peptide pool (FIG. 15). OIW-adjuvanted RBD-NP demonstrated greater expression of interferon-γ (IFNγ), TNF, and IL-2 over non-adjuvanted RBD-NP among CD4+ T cells in young mice while AddaVax and AddaS03-adjuvanted RBD-NP vaccines did not (FIG. 7A). Among aged mice, OIW and AS01B-adjuvanted RBD-NP both elicited high TNF and IL-2 responses in CD4+ T cells, while IFNγ response was generally suppressed in this population (FIG. 7C). Contrary to CD4+ T cells, limited CD8+ T cell responses were observed across treatment and age groups (FIGS. 7B and 7D).

### Example 6—OIW Adjuvants Enhance Antigen Retention and Induce Expression of Pro-Inflammatory Genes

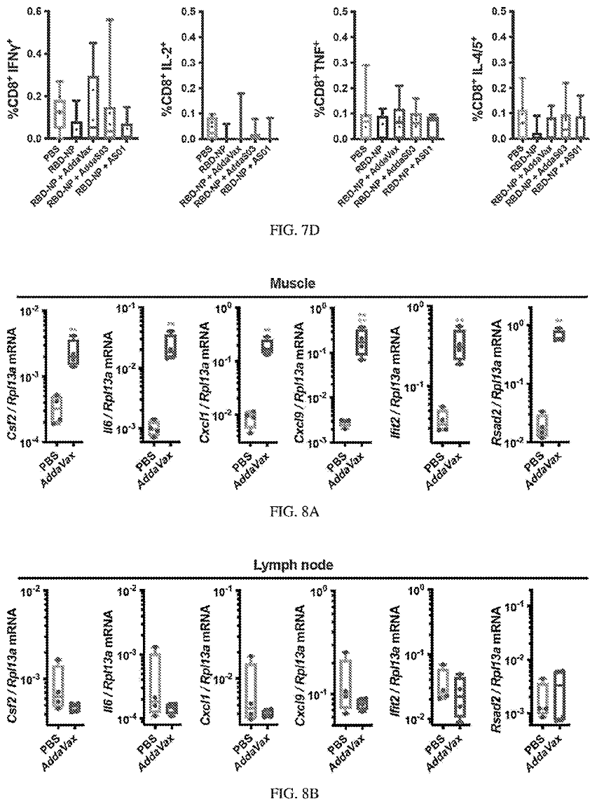

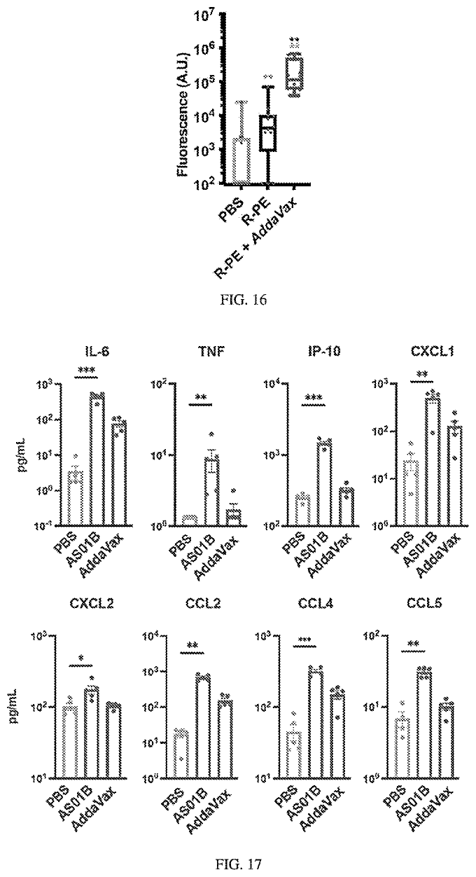

OIW emulsions are highly effective adjuvants and act through multiple mechanisms, including: 1) induction of a pro-inflammatory milieu at the injection site (Mosca et al., 2008); and/or 2) antigen targeting to and retention in the draining lymph nodes (dLN) (Cantisani et al., 2015). It was therefore assessed whether the efficacy of OIW adjuvants in RBD-NP formulations could be explained by either of these two mechanisms. To this end, mice were injected with R-Phycoerythrin (R-PE) as a model protein antigen with intrinsic fluorescence, alone or formulated with AddaVax as benchmark adjuvant. 24 hours post-injection, AddaVax promoted significant antigen retention in the dLN (FIG. 16). Interestingly, AddaVax induced high gene expression of pro-inflammatory cytokines (Csf2, Il6, Cxcl1) and interferon-stimulated genes (ISGs, e.g., Cxcl9, Ifit2, Rsad2) at the injection site (FIG. 8A-8B). Serum cytokine and chemokine concentrations were measured as a metric for systemic reactogenicity. Significant production of multiple cytokines and chemokines was observed in the serum of mice treated with AS01B, which is known for an acceptable safety profile in humans (Didierlaurent et al., 2017; Nanishi et al., 2020) but not in OIW treated mice (FIG. 17).

### Methods

### Protein Expression and Purification:

Full-length SARS-CoV-2 spike glycoprotein (M1-Q1208, GenBank MN90894) and RBD constructs (amino acid residues R319-K529, GenBank MN975262.1), both with an HRV3C protease cleavage site, a TwinStrepTag and an 8×HisTag at C-terminus, were obtained from Barney S. Graham (NIH Vaccine Research Center) and Aaron Schmidt (Ragon Institute), respectively. To generate RBD-Catch and LuS-Tag constructs in a mammalian expression vector, SARS-CoV-2 RBD and SpyCatcher (Brune et al., 2016) were fused by a GGSGGS linker for RBD-Catch, and N-terminal Spy-tag was added to lumazine synthase from Aquifex aeolicus bearing D71N mutation for LuS-Tag. Both constructs contain a signal peptide (MKHLWFFLLLVAAPRWVLS) at N-terminus and HRV3C protease site, followed by a TwinStrepTag at C-terminus. These mammalian expression vectors were used to transfect Expi293F suspension cells (Thermo Fisher) using polyethylenimine (Polysciences). Cells were allowed to grow at 37° C., 8% CO₂ for additional 5 days before harvesting for purification. Protein was purified in a PBS buffer (pH 7.4) from filtered supernatants by using either StrepTactin resin (IBA) or Cobalt-TALON resin (Takara). Affinity tags were cleaved off from eluted protein samples by HRV 3C protease, and tag removed proteins were further purified by size-exclusion chromatography using a Superose 6 10/300 column (Cytiva) for full-length Spike and a Superdex 200 10/300 Increase 10/300 GL column (Cytiva) for RBD, RBD-Catch, and LuS-Tag in a PBS buffer (pH 7.4).

### RBD-Catch and LuS-Tag Conjugations:

To saturate Lus-Tag surface with RBD, a 1:1.2 molar ratio of LuS-Tag and RBD-Catch components were mixed at 40 μM of LuS-Tag in a PBS buffer (pH 7.4) and incubated at room temperature for approximately 1 hour. Reaction mixture was applied to a Superdex200 Increase 10/300 GL column (Cytiva) in a PBS buffer (pH 7.4) to purify RBD-nanoparticles from unconjugated RBD-Catch. The conjugated RBD nanoparticle product was confirmed by SDS-PAGE and analyzed by negative-stain EM.

### SDS-PAGE Analysis:

Protein samples (250 μg/ml) in NuPAGE LDS Sample Buffer (Invitrogen) were heated to 95° C. for 5 min, and 10 μl (2.5 μg) were loaded to a NuPAGE 10% Bis-Tris gel (Invitrogen). The gel was run in NuPAGE MOPS Buffer (Invitrogen) at 60 V for 45 minutes and then 110 V for 105 min. The gel was then rinsed with DI water and fixed for 15 minutes in 50 mL of 40% ethanol and 10% acetic acid fixing solution. The gel was rinsed with DI water and incubated with QC Colloidal Coomassie Blue (Bio-Rad) on a rotating shaker for 1 hour at RT. The gel was rinsed twice with deionized water and incubated on a rotating shaker for 75 min, changing the water every 15 min, and then imaged. PNGase F kit was employed per the manufacturer's protocols (New England BioLabs). Briefly, 3.5 μg of protein samples were diluted with 1 μl Glycoprotein Denaturing Buffer and 5.5 μl DI water to a total volume of 10 μl. Samples were then heated to 100° C. for 10 minutes and chilled on ice. 2 μl of GlycoBuffer 2, 2 μl of NP-40, and 6 μl of DI water were added, followed by 1 μl of PNGase F. In non-PNGase F controls, DI water was added in place of PNGase F. Samples were incubated at 37° C. for 2 hours and then prepared as before with LDS Sample Buffer, at a final concentration of 125 μg/ml. Gels were run as before but with 1.25 μg of protein loaded per well.

### Negative Staining Electron Microscopy:

Purified RBD-nanoparticle samples were diluted to 0.01-0.05 mg/mL with a PBS (pH 7.4) buffer. A 4-μl drop of the diluted sample was applied to a freshly glow-discharged carbon-coated copper grid (400-mesh, EMS) for approximately one minute. The drop was removed using blotting paper, and the grid was washed three times with 5-μl drops of the same buffer. Adsorbed proteins were negatively stained by soaking in 4-μl drops of 2% uranyl acetate for approximately seconds and removing drop with filter paper. Micrographs were collected using JEM-1400 Plus electron microscope (JEOL, USA) operated at 80 kV, resulting in ˜0.15 nm/pixel at 80,000× magnification.

### Dynamic Light Scattering:

Purified protein samples (250 μg/ml) were loaded into a disposable microcuvette and measured at 25° C. using a Zetasizer Ultra instrument (Malvern Panalytical) equipped with a 633-nm laser with 3 scans of 60 seconds each. Each sample was measured in triplicate, and the intensity of the size distribution was plotted in GraphPad Prism 9 (GraphPad Software).

### Enzyme-Linked Immunosorbent Assay (ELISA):

ELISA was employed to examine the binding ability of the purified proteins to hACE2 and RBD-specific monoclonal Abs (mAbs). Briefly, RBD monomer, Spike trimer, RBD-Catch, LuS-Tag, and RBD-NPs were respectively diluted to concentrations of 0.5 and 5 μg/ml for mAb binding and 1 μg/ml for hACE2 binding, and 50 μl/well were added to coat 96-well high-binding flat-bottom plate (Corning) overnight at 4° C. Plates were washed with 0.05% Tween 20 PBS (PBS-T) and blocked with 1% BSA PBS for 1 hour at room temperature (RT). The plates were then incubated with sequentially 1:5 serially diluted hACE2-Fc (InvivoGen) and two anti-SARS-CoV-2 RBD mAbs (clones H4 [InvivoGen] and CR3022 [Abcam]) starting at 10 μg/ml in blocking buffer. After 2 hours of incubation, the plates were washed with PBS-T three times and incubated with HRP conjugated detection Abs (mouse anti-human IgG1 Fc-HRP, Southern Biotec). Plates were washed five times and developed with tetramethylbenzidine (OptEIA Substrate Solution, BD Biosciences) for 5 min, then stopped with 2 M H₂SO₄. Optical densities (ODs) were read at 450 nm with a SpectraMax iD3 microplate reader (Molecular Devices).

### RBD-NP Stability Analysis:

RBD-NP samples (1 mg/ml) were subjected to one to five cycles of freeze-thaw cycles by storing in a −80° C. freezer for at least 1 day, followed by incubation at RT for 30 min. For the storage temperature study, RBD-NP samples (1 mg/ml) were incubated at 4° C. or RT for 5-7 days. The RBD-NP samples were then analyzed by ELISA.

### Live SARS-CoV-2 In Vitro Competition Assay:

The day prior to infection, 5e3 VeroE6 cells were plated per well in DMEM (Quality Biological) supplemented with 10% v/v fetal bovine serum (Gibco), 1% v/v Penicillin-Streptomycin (Gemini-Bio), and 1% v/v L-Glutamine (Gibco). 1 mg/ml stock concentrations of SARS-CoV-2 Spike, SARS-CoV-2 RBD, and SARS-CoV-2 RBD-NP were diluted to 50 μg/ml in 400 μl complete VeroE6 media in a 96-well dilution block in duplicate and then serially diluted down the plate 1:3 to produce an 8-point dilution curve (125 μl into 250 μl media). Media was removed from the VeroE6 cells, and 90 μl of each dilution was then transferred to the cells and left to incubate at 37° C. and 5% CO2 for 2 hours. After incubation, each well was infected with a 0.1 M.O.I. of SARS-CoV-2 ΔORF7a::GFP (provided by Dr. Ralph Baric (UNC)) diluted in 10 μl media. A parallel plate was left uninfected to monitor cytotoxicity. After 48 hours, the infected plates were fixed in 4% paraformaldehyde for 1 hour, Hoechst-stained, and read on a plate reader (Nexcelom Biosciences, Lawrence, MA). The percentage of GFP+ cells in each well was counted and compared to an untreated, infected control to give an inhibitory concentration of 50 (IC50) for each protein. The parallel cytotoxicity plate was analyzed with Cell Titer Glo (Promega, Madison, WI) and read on a BioTek Synergy HTX plate reader (BioTek Instruments, Inc., Winooski, VT). Cell viability was compared to the untreated control.

### Animals:

Female, 3-month-old BALB/c and C57BL/6J mice were purchased from Jackson Laboratory (Bar Harbor, ME), and CD-1 mice were purchased from Charles River Laboratories (Wilmington, MA). Female, 11-13 months old BALB/c mice purchased from Taconic Biosciences (Germantown, NY) were used for aged mice experiments. Female, 6-8 weeks old wild-type (#000664) and Myd88−/− (#009088) C57BL/6 were mice purchased from The Jackson Laboratory (Bar Harbor, ME). Mice were housed under specific pathogen-free conditions at Boston Children's Hospital, and all procedures were approved under the Institutional Animal Care and Use Committee (IACUC) and operated under the supervision of the Department of Animal Resources at Children's Hospital (ARCH) (Protocol number 19-02-3897R). At the University of Maryland School of Medicine, mice were housed in a biosafety level 3 (BSL3) facility for all SARS-CoV-2 infections with all procedures approved under the IACUC (Protocol number #1120004).

### Mouse Immunization:

All formulations for immunization were prepared under sterile conditions. Mice were injected with antigens (RBD monomer, Spike trimer, and RBD-NPs), with or without adjuvants. Mock treatment mice received phosphate-buffered saline (PBS) alone. Injections (50 μl) were administered intramuscularly in the caudal thigh on Days 0 and 14. The adjuvants and their doses used were: AddaVax (25 μl), AddaS03 (25 μl) (InvivoGen), and AS01B (40 μl) (obtained from the Shingrix vaccine, GS K Biologicals SA, Belgium).

### Mouse Serum Antibody ELISA:

RBD- and Spike-specific Ab titers were quantified in serum samples by ELISA by modifying a previously described protocol (Borriello et al., 2017). Briefly, high-binding flat-bottom 96-well plates (Corning) were coated with 50 ng/well RBD or 25 ng/well Spike and incubated overnight at 4° C. Plates were washed with PBS-T (PBS+0.05% Tween 20) and blocked with 1% BSA PBS for 1 hour at RT. Serum samples were serially diluted 4-fold from 1:100 up to 1:1.058 and then incubated for 2 hours at RT. Plates were washed three times and incubated for 1 hour at RT with HRP-conjugated anti-mouse IgG, IgG1, IgG2a, or IgG2c (Southern Biotech). Plates were washed five times and developed with tetramethylbenzidine (1-Step Ultra TMB-ELISA Substrate Solution, ThermoFisher, for RBD ELISA, and BD OptEIA Substrate Solution, BD Biosciences, for Spike ELISA) for 5 min, then stopped with 2 N H2SO4. Optical densities (ODs) were read at 450 nm with a SpectraMax iD3 microplate reader (Molecular Devices). End-point titers were calculated as the dilution that emitted an optical density exceeding a 3× background. An arbitrary value of 25 was assigned to samples with OD values below the limit of detection for which it was not possible to interpolate the titer.

Surrogate of Virus Neutralization Test (sVNT):

sVNT were performed to measure the degree of hACE2/RBD inhibition by immune sera, modifying a previously published protocol (Tan et al., 2020). Briefly, high-binding flat-bottom 96-well plates (Corning, NY) were coated with 100 ng/well recombinant human ACE2 (hACE2) (Sigma-Aldrich) in PBS, incubated overnight at 4° C., washed three times with PBS-T, and blocked with 1% BSA PBS for 1 hour at RT. Each serum sample was diluted at 1:160, pre-incubated with 3 ng of RBD-Fc in 1% BSA PBS for 1 hour at RT, and then transferred to the hACE2-coated plate. RBD-Fc without pre-incubation with serum samples was added as a positive control, and 1% BSA PBS without serum preincubation was added as a negative control. Plates were then washed three times and incubated with HRP-conjugated anti-human IgG Fc (Southern Biotech) for 1 hour at RT. Plates were washed five times and developed with tetramethylbenzidine (BD OptEIA Substrate Solution, BD Biosciences) for 5 min, then stopped with 2 N H2SO4. The optical density was read at 450 nm with a SpectraMax iD3 microplate reader (Molecular Devices). Percentage inhibition of RBD binding to hACE2 was calculated with the following formula: Inhibition (%)=[1−(Sample OD value−Negative Control OD value)/(Positive Control OD value−Negative Control OD value)]×100.

### Live SARS-CoV-2 Virus Neutralization Test:

All serum samples were heat-inactivated at 56° C. for 30 minutes to deactivate complement and allowed to equilibrate to RT prior to processing for neutralization titer. Samples were diluted in duplicate to an initial dilution of 1:20 followed by 1:2 serial dilutions (vaccinated samples), resulting in a 12-dilution series with each well containing 60 μl. All dilutions employed DMEM (Quality Biological), supplemented with 10% (v/v) fetal bovine serum (heat-inactivated, Gibco), 1% (v/v) penicillin/streptomycin (Gemini Bioproducts) and 1% (v/v) L-glutamine (2 mM final concentration, Gibco). Dilution plates were then transported into the BSL-3 laboratory, and 60 μl of diluted SARS-CoV-2 (WA-1, courtesy of Dr. Natalie Thornburg/CDC) inoculum was added to each well to result in a multiplicity of infection (MOI) of 0.01 upon transfer to titering plates. A non-treated, virus-only control and mock infection control were included on every plate. The sample/virus mixture was then incubated at 37° C. (5.0% CO₂) for 1 hour before transferring 100 μl to 96-well titer plates with 5e3 VeroE6 cells. Titer plates were incubated at 37° C. (5.0% CO₂) for 72 hours, followed by cytopathic effect (CPE) determination for each well in the plate. The first sample dilution to show CPE was reported as the minimum sample dilution required to neutralize >99% of the concentration of SARS-CoV-2 tested (NT99).

### Pseudovirus Neutralization Test:

The SARS-CoV-2 pseudoviruses expressing a luciferase reporter gene were generated in an approach similar to as described previously (Yu et al., 2021; Yu et al., 2020). Briefly, the packaging plasmid psPAX2 (AIDS Resource and Reagent Program), luciferase reporter plasmid pLenti-CMV Puro-Luc (Addgene), and spike protein expressing pcDNA3.1-SARS CoV-2 SΔCT of variants were co-transfected into HEK293T cells by Lipofectamine 2000 (ThermoFisher). Pseudoviruses of SARS-CoV-2 variants were generated by using WA1/2020 strain (Wuhan/WIV04/2019, GISAID accession ID: EPI_ISL_402124), B.1.1.7 variant (GISAID accession ID: EPI_ISL_601443), or B.1.351 variant (GISAID accession ID: EPI_ISL_712096). The supernatants containing the pseudotype viruses were collected 48 hours post-transfection and were purified by centrifugation and filtration with 0.45 μm filter. To determine the neutralization activity of the plasma or serum samples from participants, HEK293T-hACE2 cells were seeded in 96-well tissue culture plates at a density of 1.75×10⁴ cells/well overnight. Three-fold serial dilutions of heat-inactivated serum or plasma samples were prepared and mixed with 50 μL of pseudovirus. The mixture was incubated at 37° C. for 1 hour before adding to HEK293T-hACE2 cells. 48 hours after infection, cells were lysed in Steady-Glo Luciferase Assay (Promega) according to the manufacturer's instructions. SARS-CoV-2 neutralization titers were defined as the sample dilution at which a 50% reduction in relative light units (RLU) was observed relative to the average of the virus control wells.

### Splenocyte Restimulation, Intracellular Cytokine Staining, and Flow Cytometry:

Mouse spleens were mechanically dissociated and filtered through a 70 μm cell strainer. After centrifugation, cells were treated with 1 mL ammonium-chloride-potassium lysis buffer for 2 minutes at RT. Cells were washed and plated in a 96-well U-bottom plate (2×106/well) and incubated overnight in RPMI 1640 supplemented with 10% heat-inactivated FBS, penicillin (100 U/ml), streptomycin (100 mg/ml), 2-mercaptoethanol (55 mM), non-essential amino acids (60 mM), HEPES (11 mM), and L-Glutamine (800 mM) (all Gibco). Next day, SARS-CoV-2 spike RBD peptide pools (PM-WCPV-S-RBD-1, JPT) were added at 0.6 nmol/ml in the presence of anti-mouse CD28/49d (1 μg/mL, BD) and brefeldin A (5 μg/ml, BioLegend). After 6 hours of stimulation, cells were washed twice and were treated with Mouse Fc Block (BD) according to the manufacturer's instructions. Cells were washed and stained with Aqua Live/Dead stain (Life Technologies, 1:500) for 15 minutes at RT. Following two additional washes, cells were incubated with the following Abs for 30 minutes at 4° C.: anti-mouse CD44 [IM7, PerCP-Cy5.5, BioLegend #103032, 1:160], anti-mouse CD3 [17A2, Brilliant Violet 785, BioLegend #100232, 1:40], anti-mouse CD4 [RM4-5, APC/Fire 750, BioLegend 100568, 1:160] and anti-mouse CD8 [53-6.7, Brilliant UltraViolet 395, BD #563786, 1:80]. After washing with PBS, cells were fixed and permeabilized by using the BD Cytofix/Cytoperm kit according to the manufacturer's instructions, and were subjected to intracellular staining (30 minutes at 4° C.) using the following Abs: anti-mouse IFNγ [XMG1.2, Alexa Fluor 488, BioLegend #505813, 1:160], anti-mouse TNF [MP6-XT22, PE Cy7, BioLegend #506324, 1:160], anti-mouse IL-2 [JES6-5H4, PE, BioLegend #503808, 1:40], anti-mouse IL-4 [11B11, APC, BioLegend #504106, 1:40] and anti-mouse IL-5 [TRFK5, APC, BioLegend #504306, 1:40]. Finally, cells were fixed in 1% paraformaldehyde (Electron Microscopy Sciences) for 20 minutes at 4° C. and stored in PBS at 4° C. until acquisition. Samples were analyzed on an LSR II (BD) flow cytometer and FlowJo v10.8.1 (FlowJo LLC).

### SARS-CoV-2 Mouse Challenge Study:

Mice were anesthetized by intraperitoneal injection of 50 μL of a xylazine and ketamine mix (0.38 mg/mouse and 1.3 mg/mouse, respectively) diluted in PBS. Mice were then inoculated intranasally with 1×10³ PFU of mouse-adapted SARS-CoV-2 (MA10, courtesy of Dr. Ralph Baric (UNC)) in 50 μl divided between nares (Leist et al., 2020). Challenged mice were weighed on the day of infection and daily for up to 4 days post-infection. At 4-days post-infection, mice were sacrificed, and lungs were collected to assess virus load by plaque assay and gene expression profiles. SARS-CoV-2 lung titers were quantified by homogenizing harvested lungs in PBS (Quality Biological Inc.) using 1.0 mm glass beads (Sigma Aldrich) and a Beadruptor (Omni International Inc.). Homogenates were added to Vero E6 cells and SARS-CoV-2 virus titers were determined by counting plaque forming units (PFU) using a 6-point dilution curve. RNA was isolated from lung homogenates using a Direct-zol RNA miniprep kit (Zymo Research), according to the manufacturer's protocol. RNA concentration and purity (260/280 and 260/230 ratios) were measured by NanoDrop (ThermoFisher Scientific). For histopathology analysis, slides were prepared as 5 μm sections and stained with hematoxylin and eosin.

Analysis of Inflammatory Responses at Injection Site, dLN, and Serum:

Young (3-month old) BALB/c mice were injected with PBS, AddaVax or CMS adjuvant, and their local muscle tissue, dLN, and serum samples were harvested for subsequent analysis 24 hours later. For dLN analysis, adjuvants were injected in caudal thigh, and inguinal LNs were collected. For muscle tissue analysis, adjuvants were injected in the gastrocnemius muscle, and the whole gastrocnemius was collected. Samples were stored in RNA later (Invitrogen) for 24 hours at 4° C. and then homogenized in TRI Reagent (Zymo Research) with a bead beater. Samples were then centrifuged, and the clear supernatant was transferred to a new tube for subsequent RNA isolation. RNA was isolated from TRI Reagent samples using phenol-chloroform extraction or column-based extraction systems (Direct-zol RNA Miniprep, Zymo Research) according to the manufacturer's protocol. RNA concentration and purity (260/280 and 260/230 ratios) were measured by NanoDrop (ThermoFisher Scientific). Cytokine and chemokine concentrations in serum samples were measured using customized Milliplex mouse magnetic bead panels (Milliplex). Assays were analyzed on the Luminex FLEXMAP 3D employing xPONENT software (Luminex) and Millipore Milliplex Analyst. Data were excluded from analysis if <30 beads were recovered.

Gene Expression Analysis by qPCR:

RNA was isolated from TRI Reagent samples using phenol-chloroform extraction or column-based extraction systems (Direct-zol RNA Miniprep, Zymo Research) according to the manufacturer's protocol. RNA concentration and purity (260/280 and 260/230 ratios) were measured by NanoDrop (Thermo Fisher Scientific). Samples with an A260/A280 ratio of <1.7 were excluded for further analysis. For lymph nodes and muscles, purified RNA was analyzed for gene expression by qPCR on a CFX384 realtime cycler (Bio-rad) using pre-designed KiCqStart SYBR Green Primers (MilliporeSigma) specific for Csf2 (RM1_Csf2 and FM1_Csf2), Cxcl9 (RM1_Cxcl9 and FM1_Cxcl9), Ifit2 (RM1_Ifit2 and FM1_Ifit2), Rsad2 (RM1_Rsad2 and FM1_Rsad2), Il6 (RM1_Il6 and FM1_Il6), Cxcl1 (RM1_Cxcl1 and FM1_Cxcl1), Rpl13a (RM1_Rpl13a and FM1_Rpl13a). For lung tissues, cDNA was prepared from purified RNA with RT2 First Strand Kit, per the manufacturer's instructions (Qiagen). cDNA was quantified by qPCR on a 7300 real-time PCR system (Applied Biosystems—Life Technologies) using pre-designed SYBR Green Primers (QIAGEN) specific for Ifit2 (PPM05993A), Rsad2 (PPM26539A), Il6 (PPM03015A), and Rpl13a (PPM03694A).

Measurement of Antigen Retention within the dLN:

The antigen retention effect of indicated adjuvants was assessed with modification of a previously published protocol (Cantisani et al., 2015). Briefly, young (3-month-old) BALB/c mice were injected IM with vaccine formulation (50 μL) applying R-phycoerythrin (R-PE) as a model antigen (6 μg). 24 hours later, the dLN was collected and homogenized in water with a beadbeater. Fluorescence values were measured with SpectraMax i3x microplate reader (Molecular Devices) and expressed as arbitrary units after background (deionized water) subtraction.

### THP1 Cell Stimulation:

THP1-Dual and THP1-Dual KO-MyD reporter cells (Invivogen) were resuspended at a concentration of 100,000 cells per well in a 96-well U-bottom plate (Corning) in 200 μl RPMI-1640 media (Gibco), supplemented with 10% fetal bovine serum (Gibco), 100 IU/ml penicillin, 100 μg/ml streptomycin, 2 mM L-glutamine, and 100 μg/ml Normocin (Invivogen). Cells were incubated for 20 hours at 37° C. in a humidified incubator at 5% CO₂ with indicated treatments. After culture, plates were centrifuged at 500 g, and supernatants were removed by pipetting without disturbing the cell pellet. To assess NF-kB activity via the conjugated SEAP reporter, 20 μl of supernatant were combined with 180 μl per well of QUANTI-Blue (Invivogen) in a clear 96-well flat-bottom plate (Corning) and incubated for 3.5 to 4 hours at 37° C. Optical density was read at 630 nm with a SpectraMax iD3 microplate reader (Molecular Devices).

### Statistical Analysis:

Statistical analyses employed Prism v9.0.2 (GraphPad Software). Some datasets were analyzed after Log-transformation as indicated in the figure legends. Statistical differences between groups in datasets with one categorical variable were evaluated by two sample t test (2 groups) or one-way ANOVA (more than 2 groups) corrected for multiple comparisons. Two-way ANOVA corrected for multiple comparisons and evaluated statistical differences between groups in datasets with two categorical variables. p values ≤0.05 were considered significant.

### REFERENCES

1. Arunachalam, P. S., Walls, A. C., Golden, N., Atyeo, C., Fischinger, S., Li, C., Aye, P., Navarro, M. J., Lai, L., Edara, V. V., et al. (2021). Adjuvanting a subunit COVID-19 vaccine to induce protective immunity. Nature.
2. Blom, A. G., and Hilgers, L. A. (2004). Sucrose fatty acid sulphate esters as novel vaccine adjuvants: effect of the chemical composition. Vaccine 23, 743-754.
3. Borriello, F., Pietrasanta, C., Lai, J. C. Y., Walsh, L. M., Sharma, P., O'Driscoll, D. N., Ramirez, J., Brightman, S., Pugni, L., Mosca, F., et al. (2017). Identification and Characterization of Stimulator of Interferon Genes As a Robust Adjuvant Target for Early Life Immunization. Front Immunol 8, 1772.
4. Brune, K. D., and Howarth, M. (2018). New Routes and Opportunities for Modular Construction of Particulate Vaccines: Stick, Click, and Glue. Front Immunol 9, 1432.
5. Brune, K. D., Leneghan, D. B., Brian, I. J., Ishizuka, A. S., Bachmann, M. F., Draper, S. J., Biswas, S., and Howarth, M. (2016). Plug-and-Display: decoration of Virus-Like Particles via isopeptide bonds for modular immunization. Sci Rep 6, 19234.
6. Cantisani, R., Pezzicoli, A., Cioncada, R., Malzone, C., De Gregorio, E., D'Oro, U., and Piccioli, D. (2015). Vaccine adjuvant MF59 promotes retention of unprocessed antigen in lymph node macrophage compartments and follicular dendritic cells. J Immunol 194, 1717-1725.
7. Chen, W. H., Wei, J., Kundu, R. T., Adhikari, R., Liu, Z., Lee, J., Versteeg, L., Poveda, C., Keegan, B., Villar, M. J., et al. (2021). Genetic modification to design a stable yeast-expressed recombinant SARS-CoV-2 receptor binding domain as a COVID-19 vaccine candidate. Biochim Biophys Acta Gen Subj 1865, 129893.
8. Cohen, A. A., Gnanapragasam, P. N. P., Lee, Y. E., Hoffman, P. R., Ou, S., Kakutani, L. M., Keeffe, J. R., Wu, H. J., Howarth, M., West, A. P., et al. (2021). Mosaic nanoparticles elicit crossreactive immune responses to zoonotic coronaviruses in mice. Science 371, 735-741.
9. Corbett, K. S., Nason, M. C., Flach, B., Gagne, M., S, O. C., Johnston, T. S., Shah, S. N., Edara, V.V., Floyd, K., Lai, L., et al. (2021). Immune Correlates of Protection by mRNA-1273 Immunization against SARS-CoV-2 Infection in Nonhuman Primates. bioRxiv.
10. Cunningham, A. L., Lal, H., Kovac, M., Chlibek, R., Hwang, S. J., Diez-Domingo, J., Godeaux, O., Levin, M. J., McElhaney, J. E., Puig-Barbera, J., et al. (2016). Efficacy of the Herpes Zoster Subunit Vaccine in Adults 70 Years of Age or Older. N Engl J Med 375, 1019-1032.
11. Dai, L., Zheng, T., Xu, K., Han, Y., Xu, L., Huang, E., An, Y., Cheng, Y., Li, S., Liu, M., et al. (2020). A Universal Design of Betacoronavirus Vaccines against COVID-19, MERS, and SARS. Cell 182, 722-733 e711.
12. Dalvie, N. C., Biedermann, A. M., Rodriguez-Aponte, S. A., Naranjo, C. A., Rao, H. D., Rajurkar, M. P., Lothe, R. R., Shaligram, U. S., Johnston, R. S., Crowell, L. E., et al. (2021a). Scalable, methanol-free manufacturing of the SARS-CoV-2 receptor binding domain in engineered Komagataella phaffii. bioRxiv.
13. Dalvie, N. C., Rodriguez-Aponte, S. A., Hartwell, B. L., Tostanoski, L. H., Biedermann, A. M., Crowell, L. E., Kaur, K., Kumru, O., Carter, L., Yu, J., et al. (2021b). Engineered SARS-CoV-2 receptor binding domain improves immunogenicity in mice and elicits protective immunity in hamsters. bioRxiv.
14. De Giovanni, M., Cutillo, V., Giladi, A., Sala, E., Maganuco, C.G., Medaglia, C., Di Lucia, P., Bono, E., Cristofani, C., Consolo, E., et al. (2020). Spatiotemporal regulation of type I interferon expression determines the antiviral polarization of CD4(+) T cells. Nat Immunol 21, 321-330.
15. Garcia-Beltran, W. F., Lam, E. C., St Denis, K., Nitido, A. D., Garcia, Z. H., Hauser, B. M., Feldman, J., Pavlovic, M. N., Gregory, D. J., Poznansky, M. C., et al. (2021). Multiple SARSCoV-2 variants escape neutralization by vaccine-induced humoral immunity. Cell 184, 2372-2383 e2379.
16. Gebre, M. S., Brito, L. A., Tostanoski, L. H., Edwards, D. K., Carfi, A., and Barouch, D. H. (2021). Novel approaches for vaccine development. Cell 184, 1589-1603.
17. GlaxoSmithKline (2022). SK bioscience and GSK's adjuvanted COVID-19 vaccine candidate meets coprimary objectives in a phase III study; Biologics License Application submitted for SKYCovione™ (GBP510/GSK adjuvant) in South Korea.
18. Graham, B. S. (2020). Rapid COVID-19 vaccine development. Science 368, 945-946.
19. Graham, B. S., Gilman, M. S. A., and McLellan, J. S. (2019). Structure-Based Vaccine Antigen Design. Annu Rev Med 70, 91-104.
20. Guo, C., Peng, Y., Lin, L., Pan, X., Fang, M., Zhao, Y., Bao, K., Li, R., Han, J., Chen, J., et al. (2021). A pathogen-like antigen-based vaccine confers immune protection against SARS-CoV-2 in non-human primates. Cell Rep Med 2, 100448.
21. Gustafson, C. E., Kim, C., Weyand, C. M., and Goronzy, J. J. (2020). Influence of immune aging on vaccine responses. J Allergy Clin Immunol 145, 1309-1321.
22. Hauser, B. M., Sangesland, M., Lam, E. C., Feldman, J., Yousif, A. S., Caradonna, T. M., Balazs, A. B., Lingwood, D., and Schmidt, A. G. (2020). Engineered receptor binding domain immunogens elicit pan-coronavirus neutralizing antibodies. bioRxiv.
23. He, L., Lin, X., Wang, Y., Abraham, C., Sou, C., Ngo, T., Zhang, Y., Wilson, I. A., and Zhu, J. (2021). Single-component, self-assembling, protein nanoparticles presenting the receptor binding domain and stabilized spike as SARS-CoV-2 vaccine candidates. Sci Adv 7.
24. Heath, P. T., Galiza, E. P., Baxter, D. N., Boffito, M., Browne, D., Burns, F., Chadwick, D. R., Clark, R., Cosgrove, C., Galloway, J., et al. (2021). Safety and Efficacy of NVX-CoV2373 Covid-19 Vaccine. N Engl J Med 385, 1172-1183.
25. Hilgers, L. A. T., Platenburg, P., Bajramovic, J., Veth, J., Sauerwein, R., Roeffen, W., Pohl, M., van Amerongen, G., Stittelaar, K. J., and van den Bosch, J. F. (2017). Carbohydrate fatty acid monosulphate esters are safe and effective adjuvants for humoral responses. Vaccine 35, 3249-3255.
26. Irvine, D. J., Aung, A., and Silva, M. (2020). Controlling timing and location in vaccines. Adv Drug Deliv Rev 158, 91-115.
27. Irvine, D. J., and Read, B. J. (2020). Shaping humoral immunity to vaccines through antigen displaying nanoparticles. Curr Opin Immunol 65, 1-6.
28. Israelow, B., Mao, T., Klein, J., Song, E., Menasche, B., Omer, S. B., and Iwasaki, A. (2021). Adaptive immune determinants of viral clearance and protection in mouse models of SARS-CoV-2. Sci Immunol 6, eab14509.
29. Joyce, M. G., King, H. A. D., Elakhal-Naouar, I., Ahmed, A., Peachman, K. K., Macedo Cincotta, C., Subra, C., Chen, R. E., Thomas, P. V., Chen, W. H., et al. (2022). A SARS-CoV-2 ferritin nanoparticle vaccine elicits protective immune responses in nonhuman primates. Sci Transl Med 14, eabi5735.
30. Katz, I. T., Weintraub, R., Bekker, L. G., and Brandt, A. M. (2021). From Vaccine Nationalism to Vaccine Equity—Finding a Path Forward. N Engl J Med 384, 1281-1283.
31. Keech, C., Albert, G., Cho, I., Robertson, A., Reed, P., Neal, S., Plested, J. S., Zhu, M., Cloney-Clark, S., Zhou, H., et al. (2020). Phase 1-2 Trial of a SARS-CoV-2 Recombinant Spike Protein Nanoparticle Vaccine. N Engl J Med 383, 2320-2332.
32. King, H. A. D., Gordon Joyce, M., Naouar, I.E., Ahmed, A., Cincotta, C. M., Subra, C., Peachman, K. K., Hack, H.H., Chen, R. E., Thomas, P. V., et al. (2021). Efficacy and breadth of adjuvanted SARS-CoV-2 receptor-binding domain nanoparticle vaccine in macaques. bioRxiv.
33. Koff, W.C., Schenkelberg, T., Williams, T., Baric, R. S., McDermott, A., Cameron, C. M., Cameron, M. J., Friemann, M. B., Neumann, G., Kawaoka, Y., et al. (2021). Development and deployment of COVID-19 vaccines for those most vulnerable. Sci Transl Med 13.
34. Kuehn, B. M. (2022). New COVID-19 Vaccine Aims to Increase Global Vaccine Access. Jama 327, 614.
35. Kuzmina, A., Khalaila, Y., Voloshin, O., Keren-Naus, A., Boehm-Cohen, L., Raviv, Y., Shemer-Avni, Y., Rosenberg, E., and Taube, R. (2021). SARS-CoV-2 spike variants exhibit differential infectivity and neutralization resistance to convalescent or post-vaccination sera. Cell Host Microbe 29, 522-528 e522.
36. Kwong, P. D., DeKosky, B. J., and Ulmer, J. B. (2020). Antibody-guided structure-based vaccines. Semin Immunol 50, 101428.
37. Lal, H., Cunningham, A. L., Godeaux, O., Chlibek, R., Diez-Domingo, J., Hwang, S. J., Levin, M. J., McElhaney, J. E., Poder, A., Puig-Barbera, J., et al. (2015). Efficacy of an adjuvanted herpes zoster subunit vaccine in older adults. N Engl J Med 372, 2087-2096.
38. Lan, J., Ge, J., Yu, J., Shan, S., Zhou, H., Fan, S., Zhang, Q., Shi, X., Wang, Q., Zhang, L., et al. (2020). Structure of the SARS-CoV-2 spike receptor-binding domain bound to the ACE2 receptor. Nature 581, 215-220.
39. Lancet Commission on, C.-V., and Therapeutics Task Force, M. (2021). Urgent needs of low income and middle-income countries for COVID-19 vaccines and therapeutics. Lancet 397, 562-564.
40. Leist, S. R., Dinnon, K. H., 3rd, Schafer, A., Tse, L. V., Okuda, K., Hou, Y. J., West, A., Edwards, C. E., Sanders, W., Fritch, E. J., et al. (2020). A Mouse-Adapted SARS-CoV-2 Induces Acute Lung Injury and Mortality in Standard Laboratory Mice. Cell 183, 1070-1085 e1012.
41. Lopez-Sagaseta, J., Malito, E., Rappuoli, R., and Bottomley, M. J. (2016). Self-assembling protein nanoparticles in the design of vaccines. Comput Struct Biotechnol J 14, 58-68.
42. Ma, X., Zou, F., Yu, F., Li, R., Yuan, Y., Zhang, Y., Zhang, X., Deng, J., Chen, T., Song, Z., et al. (2020). Nanoparticle Vaccines Based on the Receptor Binding Domain (RBD) and Heptad Repeat (HR) of SARS-CoV-2 Elicit Robust Protective Immune Responses. Immunity 53, 1315-1330 e1319.
43. McMahan, K., Yu, J., Mercado, N.B., Loos, C., Tostanoski, L. H., Chandrashekar, A., Liu, J., Peter, L., Atyeo, C., Zhu, A., et al. (2021). Correlates of protection against SARS-CoV-2 in rhesus macaques. Nature 590, 630-634.
44. Mejia, R., Hotez, P., and Bottazzi, M. E. (2020). Global COVID-19 Efforts as the Platform to Achieving the Sustainable Development Goals. Curr Trop Med Rep, 1-5.
45. Mohan, T., Zhu, W., Wang, Y., and Wang, B. Z. (2018). Applications of chemokines as adjuvants for vaccine immunotherapy. Immunobiology 223, 477-485.
46. Mosca, F., Tritto, E., Muzzi, A., Monaci, E., Bagnoli, F., Iavarone, C., O'Hagan, D., Rappuoli, R., and De Gregorio, E. (2008). Molecular and cellular signatures of human vaccine adjuvants. Proc Natl Acad Sci USA 105, 10501-10506.
47. Nanishi, E., Dowling, D. J., and Levy, O. (2020). Toward precision adjuvants: optimizing science and safety. Curr Opin Pediatr 32, 125-138.
48. O'Hagan, D. T., Lodaya, R. N., and Lofano, G. (2020). The continued advance of vaccine adjuvants—‘we can work it out’. Semin Immunol 50, 101426.
49. Piccoli, L., Park, Y. J., Tortorici, M. A., Czudnochowski, N., Walls, A. C., Beltramello, M., Silacci-Fregni, C., Pinto, D., Rosen, L. E., Bowen, J. E., et al. (2020). Mapping Neutralizing and Immunodominant Sites on the SARS-CoV-2 Spike Receptor-Binding Domain by Structure-Guided High-Resolution Serology. Cell 183, 1024-1042 e1021.
50. Pollet, J., Chen, W. H., Versteeg, L., Keegan, B., Zhan, B., Wei, J., Liu, Z., Lee, J., Kundu, R., Adhikari, R., et al. (2021). SARSCoV-2 RBD219-N1C1: A yeast-expressed SARS-CoV-2 recombinant receptor-binding domain candidate vaccine stimulates virus neutralizing antibodies and T-cell immunity in mice. Hum Vaccin Immunother, 1-11.
51. Premkumar, L., Segovia-Chumbez, B., Jadi, R., Martinez, D. R., Raut, R., Markmann, A., Cornaby, C., Bartelt, L., Weiss, S., Park, Y., et al. (2020). The receptor binding domain of the viral spike protein is an immunodominant and highly specific target of antibodies in SARSCoV-2 patients. Sci Immunol 5.
52. Pulendran, B., P, S. A., and O'Hagan, D. T. (2021). Emerging concepts in the science of vaccine adjuvants. Nat Rev Drug Discov. Richmond, P., Hatchuel, L., Dong, M., Ma, B., Hu, B., Smolenov, I., Li, P., Liang, P., Han, H.H., Liang, J., et al. (2021). Safety and immunogenicity of S-Trimer (SCB-2019), a protein subunit vaccine candidate for COVID-19 in healthy adults: a phase 1, randomised, doubleblind, placebo-controlled trial. Lancet 397, 682-694.
53. Richmond, P., Hatchuel, L., Dong, M., Ma, B., Hu, B., Smolenov, I., Li, P., Liang, P., Han, H.H., Liang, J., and Clemens, R. (2021). Safety and immunogenicity of S-Trimer (SCB-2019), a protein subunit vaccine candidate for COVID-19 in healthy adults: a phase 1, randomised, double-blind, placebo-controlled trial. Lancet 397, 682-694.
54. Sahin, U., Muik, A., Derhovanessian, E., Vogler, I., Kranz, L. M., Vormehr, M., Baum, A., Pascal, K., Quandt, J., Maurus, D., et al. (2020). COVID-19 vaccine BNT162b1 elicits human antibody and TH1 T cell responses. Nature 586, 594-599.
55. Saunders, K. O., Lee, E., Parks, R., Martinez, D. R., Li, D., Chen, H., Edwards, R.J., Gobeil, S., Barr, M., Mansouri, K., et al. (2021). SARS-CoV-2 vaccination induces neutralizing antibodies against pandemic and pre-emergent SARS-related coronaviruses in monkeys. bioRxiv.
56. Shen, X., Tang, H., Pajon, R., Smith, G., Glenn, G. M., Shi, W., Korber, B., and Montefiori, D.C. (2021). Neutralization of SARS-CoV-2 Variants B.1.429 and B.1.351. N Engl J Med. Singh, A. (2021). Eliciting B cell immunity against infectious diseases using nanovaccines. Nat Nanotechnol 16, 16-24.
57. Singh, A. (2021). Eliciting B cell immunity against infectious diseases using nanovaccines. Nat Nanotechnol 16, 16-24.
58. Sridhar, S., Joaquin, A., Bonaparte, M. I., Bueso, A., Chabanon, A. L., Chen, A., Chicz, R. M., Diemert, D., Essink, B. J., Fu, B., et al. (2022). Safety and immunogenicity of an AS03-adjuvanted SARS-CoV-2 recombinant protein vaccine (CoV2 preS dTM) in healthy adults: interim findings from a phase 2, randomised, dose-finding, multicentre study. Lancet Infect Dis 22, 636-648.
59. Tan, A. T., Linster, M., Tan, C. W., Le Bert, N., Chia, W. N., Kunasegaran, K., Zhuang, Y., Tham, C. Y. L., Chia, A., Smith, G. J. D., et al. (2021a). Early induction of functional SARS-CoV-2-specific T cells associates with rapid viral clearance and mild disease in COVID-19 patients. Cell Rep 34, 108728.
60. Tan, C. W., Chia, W. N., Qin, X., Liu, P., Chen, M. I., Tiu, C., Hu, Z., Chen, V. C., Young, B. E., Sia, W. R., et al. (2020). A SARS-CoV-2 surrogate virus neutralization test based on antibody mediated blockage of ACE2-spike protein-protein interaction. Nat Biotechnol 38, 1073-1078.
61. Tan, T. K., Rijal, P., Rahikainen, R., Keeble, A. H., Schimanski, L., Hussain, S., Harvey, R., Hayes, J. W. P., Edwards, J. C., McLean, R. K., et al. (2021). A COVID-19 vaccine candidate using SpyCatcher multimerization of the SARS-CoV-2 spike protein receptor-binding domain induces potent neutralising antibody responses. Nat Commun 12, 542.
62. Tian, J. H., Patel, N., Haupt, R., Zhou, H., Weston, S., Hammond, H., Logue, J., Portnoff, A. D., Norton, J., Guebre-Xabier, M., et al. (2021). SARS-CoV-2 spike glycoprotein vaccine candidate NVX-CoV2373 immunogenicity in baboons and protection in mice. Nat Commun 12, 372.
63. Walls, A. C., Fiala, B., Schafer, A., Wrenn, S., Pham, M. N., Murphy, M., Tse, L. V., Shehata, L., O'Connor, M. A., Chen, C., et al. (2020a). Elicitation of Potent Neutralizing Antibody Responses by Designed Protein Nanoparticle Vaccines for SARS-CoV-2. Cell 183, 1367-1382 e1317.
64. Walls, A. C., Park, Y. J., Tortorici, M. A., Wall, A., McGuire, A. T., and Veesler, D. (2020b). Structure, Function, and Antigenicity of the SARS-CoV-2 Spike Glycoprotein. Cell 181, 281-292 e286.
65. Walsh, E. E., Frenck, R. W., Jr., Falsey, A. R., Kitchin, N., Absalon, J., Gurtman, A., Lockhart, S., Neuzil, K., Mulligan, M. J., Bailey, R., et al. (2020). Safety and Immunogenicity of Two RNA Based Covid-19 Vaccine Candidates. N Engl J Med 383, 2439-2450.
66. Ward, A. B., and Wilson, I. A. (2020). Innovations in structure-based antigen design and immune monitoring for next generation vaccines. Curr Opin Immunol 65, 50-56.
67. Wrapp, D., Wang, N., Corbett, K. S., Goldsmith, J. A., Hsieh, C. L., Abiona, O., Graham, B. S., and McLellan, J. S. (2020). Cryo-EM structure of the 2019-nCoV spike in the prefusion conformation. Science 367, 1260-1263.
68. Wu, K., Choi, A., Koch, M., Elbashir, S., Ma, L., Lee, D., Woods, A., Henry, C., Palandjian, C., Hill, A., et al. (2021). Variant SARS-CoV-2 mRNA vaccines confer broad neutralization as primary or booster series in mice. bioRxiv.
69. Yan, R., Zhang, Y., Li, Y., Xia, L., Guo, Y., and Zhou, Q. (2020). Structural basis for the recognition of SARS-CoV-2 by full-length human ACE2. Science 367, 1444-1448.
70. Yang, J., Wang, W., Chen, Z., Lu, S., Yang, F., Bi, Z., Bao, L., Mo, F., Li, X., Huang, Y., et al. (2020). A vaccine targeting the RBD of the S protein of SARS-CoV-2 induces protective immunity. Nature 586, 572-577.
71. Yu, J., Li, Z., He, X., Gebre, M. S., Bondzie, E. A., Wan, H., Jacob-Dolan, C., Martinez, D. R., Nkolola, J.P., Baric, R. S., and Barouch, D. H. (2021). Deletion of the SARS-CoV-2 Spike Cytoplasmic Tail Increases Infectivity in Pseudovirus Neutralization Assays. J Virol. 10.1128/JVI.00044-21.
72. Yu, J., Tostanoski, L. H., Peter, L., Mercado, N.B., McMahan, K., Mahrokhian, S. H., Nkolola, J.P., Liu, J., Li, Z., Chandrashekar, A., et al. (2020). DNA vaccine protection against SARS-CoV-2 in rhesus macaques. Science 369, 806-811. 10.1126
73. Zhang, X., Meining, W., Fischer, M., Bacher, A., and Ladenstein, R. (2001). X-ray structure analysis and crystallographic refinement of lumazine synthase from the hyperthermophile Aquifex aeolicus at 1.6 A resolution: determinants of thermostability revealed from structural comparisons. J Mol Biol 306, 1099-1114.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A nanoparticle comprising a multimeric protein scaffold comprising a lumazine synthase and a protein antigen from a Beta coronavirus.

2. The nanoparticle of claim 1, wherein the lumazine synthase is from Aquifex aeolicus.

3. The nanoparticle of claim 1 or claim 2, wherein the multimeric protein scaffold comprises at least 60 subunits of lumazine synthase.

4. The nanoparticle of any one of claims 1-3, wherein the Beta coronavirus is Middle East Respiratory Syndrome coronavirus (MERS-CoV), Severe Acute Respiratory Syndrome (SARS)-associated coronavirus 1 (SARS-CoV-1), or SARS-CoV-2.

5. The nanoparticle of claim 4, wherein the protein antigen is a MERS-CoV spike protein, a SARS-CoV-1 spike protein, or a SARS-CoV-2 spike protein.

6. The nanoparticle of claim 4, wherein the protein antigen comprises a protein domain from a MERS-CoV, SARS-CoV-1, or SARS-CoV-2 protein.

7. The nanoparticle of claim 6, wherein the protein antigen is a MERS-CoV spike protein receptor binding domain (RBD), a SARS-CoV-1 spike protein RBD, or a SARS-CoV-2 spike protein RBD.

8. The nanoparticle of any one of claims 1-7, wherein the lumazine synthase and the protein antigen are covalently linked.

9. The nanoparticle of claim 8, wherein the lumazine synthase and the protein antigen are linked through a covalent bond formed between SEQ ID NO: 3 and SEQ ID NO: 4.

10. The nanoparticle of anyone of claims 1-9, wherein the protein antigen is displayed on the surface of the nanoparticle.

11. The nanoparticle of any one of claims 1-10, wherein the nanoparticle enhances an immune response against the protein antigen when administered to a subject, compared to when the protein antigen is administered alone.

12. The nanoparticle of claim 11, wherein the nanoparticle enhances the production of antigen-specific antibodies when administered to the subject, compared to when the protein antigen is administered alone.

13. The nanoparticle of claim 12, wherein the antigen-specific antibodies comprise immunoglobulin G (IgG).

14. The nanoparticle of claim 13, wherein the IgG is a subclass 1 IgG (IgG1) or a subclass 2 IgG (IgG2).

15. The nanoparticle of any one of claims 12-14, wherein the antigen-specific antibodies are neutralizing antibodies against a variant of MERS-CoV, SARS-CoV-1, or SARS-CoV-2.

16. The nanoparticle of claim 15, wherein the antigen specific antibodies are neutralizing antibodies against wild-type SARS-CoV-2, B.1.1.7 SARS-CoV-2, or B.1.351 SARS-CoV-2.

17. The nanoparticle of any one of claims 11-16, wherein the nanoparticle prolongs a protective effect in the subject against the Beta coronavirus protein antigen, compared to when the Beta coronavirus protein antigen is administered alone.

18. The nanoparticle of any one of claims 11-17, wherein the subject is a human.

19. A composition comprising the nanoparticle of any one of claims 1-18.

20. The composition of claim 19, wherein the composition further comprises a pharmaceutically acceptable carrier.

21. A composition comprising the nanoparticle of any one of claims 1-18 and a squalene-based oil in water emulsion (OIW) or a liposomal adjuvant.

22. The composition of claim 21, wherein the squalene-based OIW comprises an emulsion of sorbitan trioleate, squalene, and polysorbate 80 or an emulsion of DL-α-tocopherol, squalene, and polysorbate 80.

23. The composition of claim 21, wherein the liposomal adjuvant comprises 3-O-desacyl-4′-monophosphoryl lipid A, saponin QS-21, dioleoyl phosphatidylcholine (DOPC), and cholesterol.

24. The composition of any one of claims 21-23, wherein the composition further comprises a second adjuvant.

25. The composition of claim 24, wherein the second adjuvant is a Toll-like receptor (TLR) agonist.

26. The composition of any one of claims 21-25, wherein the composition is a vaccine composition.

27. A method of enhancing an immune response in a subject in need thereof, the method comprising administering to the subject an effective amount of the nanoparticle of any one of claims 1-18, the composition of claim 19 or claim 20, or the composition of any one of claims 21-26.

28. A method of treating a disease or reducing the risk of a disease, the method comprising administering to a subject in need thereof an effective amount of the nanoparticle of any one of claims 1-18, the composition of claim 19 or claim 20, or the composition of any one of claims 21-26.

29. The method of claim 28, wherein the disease is a disease caused by a Beta coronavirus.

30. The method of claim 29, wherein the disease is Middle East Respiratory Syndrome (MERS), Sudden Acute Respiratory Syndrome (SARS), or Coronavirus Disease 2019 (COVID-19).

31. The method of any one of claims 27-30, wherein the subject is a human neonate, an infant, an adult, or an elderly individual.

32. The method of claim 31, wherein the subject is a human adult.

33. The method of claim 31, wherein the subject is an elderly human.

34. The method of claim 33, wherein the administration occurs when the subject is more than 65 years of age.

35. The method of claim 31, wherein the subject is immunocompromised, immunosenescent, has a chronic illness, is malnourished, or is frail.

36. The method of any one of claims 27-35, wherein the administration is intravenous, intramuscular, intradermal, oral, topical, intranasal, or sublingual.

37. The method of any one of claims 27-36, wherein the administration occurs more than once.

38. The method of any one of claims 27-37, wherein the administration elicits an immune response to a Beta coronavirus protein antigen in the subject.

39. The method of claim 38, wherein the immune response comprises an innate immune response.

40. The method of claim 38, wherein the immune response comprises an adaptive immune response.

41. The method of any one of claims 38-40, wherein the administration elicits the production of one or more pro-inflammatory cytokines in the subject.

42. The method of claim 41, wherein the pro-inflammatory cytokines are selected from one or more of CSF-2, IL-6, and CXCL1.

43. The method of any one of claims 38-42, wherein the administration elicits the expression of one or more interferon (IFN)-stimulated genes in the subject.

44. The method of claim 43, wherein the IFN-stimulated genes comprise type I IFN-stimulated genes.

45. The method of claim 43 or claim 44, wherein the IFN-stimulated genes are selected from one or more of CXCL19, IFIT2, and RSAD2.

46. The method of any one of claims 38-45, wherein the administration enhances antigen retention in draining lymph nodes of the subject.

47. The method of any one of claims 38-46, wherein the administration elicits the production of antigen-specific antibodies in the subject.

48. The method of claim 47, wherein the antigen-specific antibodies comprise immunoglobulin G (IgG).

49. The method of claim 48, wherein the IgG is a subclass 1 IgG (IgG1) or a subclass 2 IgG (IgG2).

50. The method of any one of claims 47-49, wherein the antigen-specific antibodies are neutralizing antibodies against a variant of MERS-CoV, SARS-CoV-1, or SARS-CoV-2.

51. The method of claim 50, wherein the antigen specific antibodies are neutralizing antibodies against wild-type SARS-CoV-2, B.1.1.7 SARS-CoV-2, or B.1.351 SARS-CoV-2.
